SEQUENCE LISTING (22 sequences)

SEQ ID NO: 1 - protein, 491 aa, Homo sapiens [sequence omitted]

SEQ ID NO: 2 - protein, 112 aa, Homo sapiens
  MISC_FEATURE: AB 1 heavy chain variable region (VH)
QVQLVQSGAEVTKPGASVKVSCKASGYTFTSYWMQWVRQAPGQGLEWVGEIDPSNSYTNYNQKFRGRVTLTRDTSTTTVYMELSSLRSEDTAVYYCARGGYWGQGTTVTVSS

SEQ ID NO: 3 - protein, 112 aa, Homo sapiens
  MISC_FEATURE: AB 1 light chain variable region (VL)
DIVMTQSPLSLPVTPGEPASISCRSSKSLLHSQGITYLYWYLQKPGQSPQLLIYQLSNRASGVPDRFSGSGSGTDFTLKISRVEAEDVGVYYCVQNLELPYTFGGGTKLEIK

SEQ ID NO: 4 - protein, 10 aa, Homo sapiens
  MISC_FEATURE: AB 1 CDR-H1
GYTFTSYWMQ

SEQ ID NO: 5 - protein, 17 aa, Homo sapiens
  MISC_FEATURE: AB 1 CDR-H2
EIDPSNSYTNYNQKFRG

SEQ ID NO: 6 - protein, 5 aa, Homo sapiens
  MISC_FEATURE: AB 1 CDR-H3
ARGGY

SEQ ID NO: 7 - protein, 16 aa, Homo sapiens
  MISC_FEATURE: AB 1 CDR-L1
RSSKSLLHSQGITYLY

SEQ ID NO: 8 - protein, 7 aa, Homo sapiens
  MISC_FEATURE: AB 1 CDR-L2
QLSNRAS

SEQ ID NO: 9 - protein, 9 aa, Homo sapiens
  MISC_FEATURE: AB 1 CDR-L3
VQNLELPYT

SEQ ID NO: 10 - protein, 120 aa, Homo sapiens
  MISC_FEATURE: AB 2 heavy chain variable region (VH)
EVQLVQSGAEVKKPGESLRISCKASGYTFTSYWMHWVRQMPGKGLEWMGEIDPSDSYTNYNQKFKGHVTISADKSISTAYLQWSSLKASDTAMYYCAKSNYVVPWYFDVWGQGTLVTVSS

SEQ ID NO: 11 - protein, 112 aa, Homo sapiens
  MISC_FEATURE: AB 2 light chain variable region (VL)
EIVLTQSPGTLSLSPGERATLSCRSSKSLLHSNGITYLYWYQQKPGQAPRLLIYQMSNLASGIPDRFSGSGSGTDFTLTISRLEPEDFAVYYCAQNLELPWTFGGGTKLEIK

SEQ ID NO: 12 - protein, 10 aa, Homo sapiens
  MISC_FEATURE: AB 2 CDR-H1
GYTFTSYWMH

SEQ ID NO: 13 - protein, 17 aa, Homo sapiens
EIDPSDSYTNYNQKFKG

SEQ ID NO: 14 - protein, 13 aa, Homo sapiens
  MISC_FEATURE: AB 2 CDR-H3
AKSNYVVPWYFDV

SEQ ID NO: 15 - protein, 16 aa, Homo sapiens
  MISC_FEATURE: AB 2 CDR-L1
RSSKSLLHSNGITYLY

SEQ ID NO: 16 - protein, 7 aa, Homo sapiens
  MISC_FEATURE: AB 2 CDR-L2
QMSNLAS

SEQ ID NO: 17 - protein, 9 aa, Homo sapiens
  MISC_FEATURE: AB 2 CDR-L3
AQNLELPWT

SEQ ID NO: 18 - DNA, 401 bp, Homo sapiens
  misc_feature: probe for detecting rs7789066
aatgtgggctttgtttatggtagtatttttttaagatgcaaaatttgatcttgcaatctttgagttgaatttgcagttttaaaataaaaaggtcttatatctgtgcaaagaaaaaatattgtattgacattgcttgttaaattaaggagtgaggcctgcacaagtattagtaatgtgaatcctcacagtagtctccagagaaaaaaaatgacaatgaagtcatgttaccaataggacaatcaccatttgcctgagatagaaataggcacattctctatgtaactacatgcttaagctggagcaattcagaattaattggggtttagaactatgaaattatcactgaaaacagagccaagatttcattttaaaatggcctcccctgaaagacagtttaacag

SEQ ID NO: 19 - DNA, 401 bp, Homo sapiens
  misc_feature: probe for detecting rs61330082
agtggaacttgtgaattgagattcatagtggaacttgtgaattgagattcatctcgaaactggaggcatggctgagacttctaataaagacaacctcagtcaacactatgtcttgaagtcagtatatatttttgacaatcacctcatctacacgtagatacaatacagggcaaagatcatggaagtggaaggtatcaccaggcactcaccaatgtagtaaatactagtacacttacaattattttcagcaacgaggtttgaaacaagagggcttatgtatttattggttgatcttccctgtgttttaccggggaaaattatttgtaaacgcatttaaacaaattattatttctattttgagacggagtctcgctctgtcgcccaggctggagtgcagtggc

SEQ ID NO: 20 - DNA, 401 bp, Homo sapiens
  misc_feature: probe for detecting rs9770242
ccgctttcctccggcggctctgtctatggctgagctctttgatcctttgagagatggtttgacttttcccgagcaaagagcctgcgttgaaaagcgggggtggaattcagtcctcacagataatgaggggacaagacctaattgaaccgagtattgccgggaaggaaaaggcaacgggccaagcctttgacagggtgcgacactgacttttatcatcgttatagtctttaaatcctgggaaacgagttggcaaccccaaaataaagaagtgtaatgacgtctgatgacttcacccaaatacagaccattccaagaaagacttgcgcagttctcatgcgtggttgcgtttttgcataaaactaagattccctttgtccgcatgtttaatagcttaaaaataa

SEQ ID NO: 21 - DNA, 401 bp, Homo sapiens
  misc_feature: probe for detecting rs59744560
gagctgcggtgaggagtgaggctgaggggcccctttcatctgatgcagcgactccgctttcctccggcggctctgtctatggctgagctctttgatcctttgagagatggtttgacttttcccgagcaaagagcctgcgttgaaaagcgggggtggaattcagtcctcacagataatgaggggacaagacctaattgaaccgagtattgccgggaaggaaaaggcaacgggccaagcctttgacagggtgcgacactgacttttatcatcgttatagtctttaaatcctgggaaacgagttggcaaccccaaaataaagaagtgtaatgacgtctgatgacttcacccaaatacagaccattccaagaaagacttgcgcagttctcatgcgtggttgcgtt

SEQ ID NO: 22 - DNA, 401 bp, Homo sapiens
  misc_feature: probe for detecting rs1319501
gggagctctggcggactccccacctcggttcccccgccttcaccccgtcaccctccgggggccgagaaagggcggggcgcggcagcgcgctgcgcagtgcgcggaggcggggcggggaggaggacgtgatgcacgcgctcttcctcccagacgccagctctgggaagctggaggcagcggggcagccccggcgcgtgacccgggcgcttacctaagttcgagttcccggcacgggcgcgggagggcggggcctggagggggcgttcccagctttgccagtgccacgaggagccggttcgcccgccccgcctgggaccttccgtcctacccagtcctggccggttttctgggtcctcctgaagtcacgccacccggctagggggcgaggagcctcctactgc

(19) United States
(12) Patent Application Publication
(10) Pub. No.: US 2022/0290246 A1
(43) Pub. Date: Sep. 15, 2022

(54) SINGLE NUCLEOTIDE POLYMORPHISMS AND USES THEREOF

(71) Applicant: Arizona Board of Regents on Behalf of the University of Arizona, Tucson, AZ (US)

(72) Inventor: JOE G.N. GARCIA, Tucson, AZ (US)

(73) Assignee: Arizona Board of Regents on Behalf of the University of Arizona, Tucson, AZ (US)

(21) Appl. No.: 17/632,950

(22) PCT Filed: Aug. 7, 2020
(86) PCT No.: PCT/US2020/045476

§ 371 Date: Feb. 4, 2022

Related U.S. Application Data
(60) Provisional application No. 62/883,934, filed on Aug. 7, 2019.

(51) Int. Cl. C12Q 1/6886; G01N 33/50; C12Q 1/6813

(52) U.S. Cl. CPC C12Q 1/6886; G01N 33/50; C12Q 1/6813; C12Q 2600/156

(57) ABSTRACT

A method of identifying single nucleotide polymorphisms (SNPs) within the NAMPT promoter that are associated with inflammatory conditions. Also provided are methods of diagnosing and treating inflammatory conditions in a subject.

### RELATED APPLICATION

This application claims priority to U.S. Provisional Application No. 62/883,934, filed on Aug. 7, 2019, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Aug. 7, 2020, is named A110808_1040WO_Seq_Listing_ST25.txt and is 15,054 bytes in size.

### FIELD OF THE INVENTION

This invention relates generally to the fields of inflammation, cancer and molecular biology. The invention provides methods of detecting single nucleotide polymorphisms (SNPs), methods for predicting the increased risk of developing inflammatory disease, cancer, and methods of determining the responsiveness of a patient (cancer or other) to a treatment, using SNPs.

### BACKGROUND

Inflammatory diseases include an array of disorders and conditions characterized by inflammation, including acute respiratory distress syndrome (ARDS), radiation-induced lung injury (RILI), pulmonary hypertension, or pulmonary fibrosis. There is a need in the art for improved methods of diagnosing and treating such conditions.

Moreover, cancer is a leading cause of morbidity and mortality in most developed countries. However, few if any specific methods for predicting cancer risk, predicting the responsiveness of the subject to a particular treatment regimen and effective treatment options after diagnosis of cancer are known and, therefore, much work has focused on improving methods addressing the aforementioned factors.

Prostate cancer (PCa) in particular represents an unmet need for therapies that halt PCa progression and recurrence. PCa is generally an indolent tumor initially after first line androgen-deprivation therapy (ADT), but often exhibits widespread recurrence 5-10 years post ADT therapy (85%). Targeting the transition from organ-confined PCa (95% survival) to invasive and metastatic cancer (30% survival) is paramount to influencing PCa lethality. Cytotoxic cancer therapies for androgen-independent primary tumors are ineffective at eradicating metastatic lesions. Current concepts of the basis for PCa transition from indolent disease to aggressive cancer phenotype that escapes from the capsule and metastasizes includes a significant role for inflammatory signaling pathways.

### SUMMARY OF THE INVENTION

Reducing the morbidity and mortality of PCa includes identification of race-specific risk factors influencing PCa glandular escape and metastatic progression; identification of race-specific biomarkers that herald this progression; and development of novel, effective, personalized approaches that attenuate this progression. The absence of novel race-specific biomarkers, the paucity of information on race-specific PCa risk factors, and the lack of effective personalized therapies are serious unmet needs to combat PCa progression and development of fatal disease.

Regulation of innate immunity and reduction of inflammatory injury associated with can tumor, particularly, regulating NFkB-dependent inflammatory cascade, may be important in both prostate cancer initiation and therapeutic resistance.

In order to improve treatment and survival of PCa, particularly inhibiting PCa progression, biomarkers are needed that are predictive as to the responsiveness of a patient to a particular therapy. Non-limiting examples of such markers include Single Nucleotide Polymorphisms (SNPs) in genes regulating cytokines such as nicotinamide phospho-ribosyltransferase enzyme (NAMPT), also called visfatin’. At least some embodiments of the present invention identify SNPs within the NAMPT promoter that are associated with transition from indolent to aggressive prostate cancer. At least some embodiments of the present invention provide a means to identify patients who may be at risk for PCa disease progression. Also provided are methods of diagnosing and treating prostate cancer in a subject.

In some embodiments, provided herein is a method of identifying a subject at risk of developing aggressive prostate cancer, comprising the steps of (a) obtaining a sample from a subject having indolent prostate cancer; and (b) detecting the presence of at least one single nucleotide polymorphism (SNP) associated with human nicotinamide phosphoribosyl transferase (NAMPT) in the sample. The at least one SNP is selected from the group consisting of rs7789066, rs61330082, rs9770242, rs59744560, rs116647506, rs1319501, rs114382471, and rs190893183.

In some embodiments, the subject has indolent prostate cancer that is inherited.

In some embodiments, the subject has at least 2 SNPs, at least 3 SNPs, at least 4 SNPs, at least 5 SNPs, at least 6 SNPs, at least 7 SNPS, or 8 SNPs selected from the group consisting of rs7789066, rs61330082, rs9770242, rs59744560, rs116647506, rs1319501, rs114382471, and rs190893183. In a specific embodiment, the method comprises detecting at least 2 SNPs selected from the group consisting of rs7789066, rs61330082, rs9770242 and rs59744560, rs116647506, rs1319501, rs114382471, and rs190893183.

In some embodiments, the method comprises detecting at least one SNP selected from the group consisting of rs7789066, rs61330082, rs9770242 and rs59744560.

In some embodiments, the method comprises detecting at least one SNP selected from the group consisting of rs116647506, rs61330082, rs114382471, and rs190893183.

In some embodiments of the method of identifying a subject at risk of developing aggressive prostate cancer, the subject is of African descent.

In some embodiments of the method of identifying a subject at risk of developing aggressive prostate cancer, the detecting comprises using a polymerase chain reaction (PCR), a SNP microarray, SNP-restriction fragment length polymorphism (SNP-RFLP), dynamic allele-specific hybridization (DASH), primer extension (MALDI-TOF) mass spectrometry, single strand conformation polymorphism, and/or new generation sequencing (NGS).

In some embodiments of the method of identifying a subject at risk of developing aggressive prostate cancer, detecting comprises contacting the sample with an oligonucleotide probe that selectively hybridizes to a nucleotide sequence comprising the SNP, or a nucleotide sequence complementary thereto, and detecting selective hybridization of the oligonucleotide probe. In certain embodiments, an oligonucleotide probe that selectively hybridizes to a nucleotide sequence comprising the SNP includes 200 base pairs on each side surrounding the SNP. In some embodiments, the oligonucleotide probe comprising the nucleotide sequence set forth in SEQ ID NO: 18 selectively hybridizes to a nucleotide sequence comprising rs7789066; an oligonucleotide probe comprising the nucleotide sequence set forth in SEQ ID NO: 19 selectively hybridizes to a nucleotide sequence comprising rs61330082; an oligonucleotide probe comprising the nucleotide sequence set forth in SEQ ID NO: 20 selectively hybridizes to a nucleotide sequence comprising rs9770242; an oligonucleotide probe comprising the nucleotide sequence set forth in SEQ ID NO: 21 selectively hybridizes to a nucleotide sequence comprising rs59744560; and/or an oligonucleotide probe comprising the nucleotide sequence set forth in SEQ ID NO: 22 selectively hybridizes to a nucleotide sequence comprising rs1319501.

In some embodiments, the oligonucleotide probe comprises a detectable label, and wherein detecting selective hybridization of the probe comprises detecting the detectable label. In specific embodiments, the detectable label comprises a fluorescent label, a luminescent label, a radionuclide, or a chemiluminescent label. In other embodiments, the oligonucleotide probe comprises a bilabeled oligonucleotide probe, comprising a fluorescent moiety and a fluorescent quencher.

In some embodiments, the method of identifying a subject at risk of developing aggressive prostate cancer further comprises detecting one or more additional SNPs associated with a NAMPT promoter activity level that is higher than a baseline NAMPT promoter activity level.

In some embodiments of the aforementioned method of identifying a subject at risk of developing aggressive prostate cancer, the sample is a plasma sample.

Some embodiments provide a method of treating a subject having indolent prostate cancer, comprising the steps of (a) obtaining a sample from a subject having indolent prostate cancer; (b) detecting the presence or absence of at least one SNP in the sample, selected from the group consisting of rs7789066, rs61330082, rs9770242, rs59744560, rs116647506, rs1319501, rs114382471, and rs190893183, and (c) administering to the subject at risk for developing aggressive prostate cancer (i) an effective amount of an eNAMPT inhibitor and/or (ii) one or more of radiation therapy (e.g., external beam radiation; and/or brachytherapy); hormone therapy such as luteinizing hormone-releasing hormone (LH-RH) agonists (e.g., leuprolide; goserelin; triptorelin; and/or histrelin) or other medications to stop the body from producing testosterone (e.g., ketoconazole; and/or abiraterone); anti-androgens (e.g., bicalutamide; nilutamide; flutamide; and/or enzalutamide); chemotherapy; and biological therapy (e.g., sipuleucel-T), such that the subject having indolent prostate cancer is treated. The presence of the at least one SNP indicates that the subject is at risk for developing aggressive prostate cancer.

In some embodiments of the method of treating a subject having indolent prostate cancer, the sample is a plasma sample.

In some embodiments of the method of treating a subject having indolent prostate cancer, the method comprises detecting at least 2 SNPs, at least 3 SNPs, at least 4 SNPs, at least 5 SNPs, at least 6 SNPs, at least 7 SNPS, or 8 SNPs selected from the group consisting of rs7789066, rs61330082, rs9770242, rs59744560, rs116647506, rs1319501, rs114382471, and rs190893183. In some embodiments, the SNP is selected from the group consisting of rs7789066, rs61330082, rs9770242 and rs59744560. In other embodiments, the SNP is selected from the group consisting of rs116647506, rs61330082, rs114382471, and rs190893183.

In some embodiments of the method of treating a subject having indolent prostate cancer, the subject is of African descent.

In some embodiments of the method of treating a subject having indolent prostate cancer, the detecting comprises using a polymerase chain reaction (PCR), a SNP microarray, SNP-restriction fragment length polymorphism (SNP-RFLP), dynamic allele-specific hybridization (DASH), primer extension (MALDI-TOF) mass spectrometry, single strand conformation polymorphism, and/or new generation sequencing (NGS). In some embodiments, the presence of the SNP is determined by contacting the sample with an oligonucleotide probe that selectively hybridizes to a nucleotide sequence comprising the SNP, or a nucleotide sequence complementary thereto, and detecting selective hybridization of the oligonucleotide probe. In certain embodiments, an oligonucleotide probe that selectively hybridizes to a nucleotide sequence comprising the SNP includes 200 base pairs on each side surrounding the SNP. In particular embodiments, an oligonucleotide probe comprising the nucleotide sequence set forth in SEQ ID NO: 18 selectively hybridizes to a nucleotide sequence comprising rs7789066; an oligonucleotide probe comprising the nucleotide sequence set forth in SEQ ID NO: 19 selectively hybridizes to a nucleotide sequence comprising rs61330082; an oligonucleotide probe comprising the nucleotide sequence set forth in SEQ ID NO: 20 selectively hybridizes to a nucleotide sequence comprising rs9770242; an oligonucleotide probe comprising the nucleotide sequence set forth in SEQ ID NO: 21 selectively hybridizes to a nucleotide sequence comprising rs59744560; and/or an oligonucleotide probe comprising the nucleotide sequence set forth in SEQ ID NO: 22 selectively hybridizes to a nucleotide sequence comprising rs1319501.

In some embodiments, the oligonucleotide probe comprises a detectable label, and wherein detecting selective hybridization of the probe comprises detecting the detectable label. In a particular embodiment, the detectable label comprises a fluorescent label, a luminescent label, a radionuclide, or a chemiluminescent label. In further embodiments, the oligonucleotide probe comprises a bilabeled oligonucleotide probe, comprising a fluorescent moiety and a fluorescent quencher.

In some embodiments of the method of treating a subject having indolent prostate cancer, the method further comprises detecting one or more additional SNPs associated with a NAMPT promoter activity level that is higher than a baseline NAMPT promoter activity level. In specific embodiments, the baseline NAMPT promoter activity level is a level associated with indolent prostate cancer.

In some embodiments of the method of treating a subject having indolent prostate cancer, the method comprises administering the eNAMPT inhibitor, wherein the eNAMPT inhibitor is an anti-eNAMPT antibody. In specific embodiments, the anti-eNAMPT antibody comprises a heavy chain comprising a variable region comprising CDR1, CDR2, and a CDR3 domains as set forth in amino acid sequences of SEQ ID Nos: 4, 5, and 6, respectively; and a light chain comprising a variable region comprising CDR1, CDR2, and a CDR3 domains as set forth in amino acid sequences of SEQ ID Nos: 7, 8, and 9, respectively. In another specific embodiment, the heavy chain variable region comprises the amino acid sequence set forth in SEQ ID NO: 2, and the light chain variable region comprises the amino acid sequence set forth in SEQ ID NO: 3. In a different embodiment, the anti-eNAMPT antibody comprises a heavy chain comprising a variable region comprising CDR1, CDR2, and a CDR3 domains as set forth in amino acid sequences of SEQ ID Nos: 12, 13, and 14, respectively; and a light chain comprising a variable region comprising CDR1, CDR2, and a CDR3 domains as set forth in amino acid sequences of SEQ ID Nos: 15, 16, and 17, respectively. In yet another embodiment, the heavy chain variable region comprises the amino acid sequence set forth in SEQ ID NO: 10, and the light chain variable region comprises the amino acid sequence set forth in SEQ ID NO: 11.

### BRIEF DESCRIPTION OF THE DRAWINGS

FIGS. 1A-1D depict immunohistochemical (IHC) staining results for NAMPT in minimally invasive prostate cancer (PCa), highly invasive PCa, or in normal prostate tissues. FIG. 1A is a representative micrograph showing extremely low NAMPT expression in normal prostate tissue. FIG. 1B is a representative micrograph showing significantly increased albeit moderate NAMPT expression in organ-confined prostatic adenocarcinoma. FIG. 1C provides representative micrographs showing strong NAMPT expression within tumor cells in two separate prostatic adenocarcinomas with smooth muscle capsular penetration and invasion into extra-prostatic space. FIG. 1D is a graph showing cumulative analysis of NAMPT expression in normal (benign) prostate tissue or in PCa patients with organ-confined and capsule invasive disease. * indicates p<0.05; ** indicates p<0.005

FIGS. 2A-2B are graphical representations of plasma NAMPT level in healthy controls, PCa patients, or high risk subjects. FIG. 2A is a graph depicting plasma NAMPT level in PCa patients (95% Confidence Interval (CI): 22.4-41)), high risk subjects (95% CI: 15.9-19.5), or healthy controls (95% CI: 12.3-17.5). FIG. 2B is a graph showing plasma NAMPT level in patients with organ-confined PCa (95% CI: 17.6-21.8) or extra-prostatic PCa (95% CI: 23.1-52.1). * indicates p<0.05; *** indicates p<0.001

FIGS. 3A-3E depicts effects of a humanized anti-NAMPT monoclonal antibody (mAb) on human PCa cell invasion, as evaluated in severe combined immunodeficient (SCID) mice. FIG. 3A is a representative micrograph showing severe studding of the peritoneum with cancer cell invasion through the smooth muscle layer (invasion sites: 26; invasion depth: 100 μm) in SCID mice after injection of PC3, a highly metastatic human PCa cell. NAMPT expression in the invading cells is markedly increased. FIG. 3B provides an enlarged image of the sites of cancer cell invasion utilizing the same SCID mouse model as described for FIG. 3A, but from different experiments FIG. 3C is a representative micrograph showing inhibition of PC3 cell invasion in PC3-challenged SCID mice that received weekly intraperitoneal (i.p.) injection of humanized anti-NAMPT mAb. FIG. 3D is a graph showing percent tumor invasion in PC3-challenged SCID mice that were injected with humanized anti-NAMPT mAb or vehicle control. FIG. 3E is a graph showing tumor invasion depth (in μm) in PC3-challenged SCID mice that were injected with humanized anti-NAMPT mAb or vehicle control. * indicates p<0.05

FIGS. 4A-4B graphically represents plasma levels of NAMPT and other inflammatory cytokines, such as IL-6, IL-8, and macrophage migration inhibitory factor (MIF) in acute respiratory distress syndrome (ARDS) and other acute inflammatory conditions. FIG. 4A is a graph depicting plasma NAMPT level in patients with COVID-19 infection, ARDS or trauma, or in healthy controls (“Ctl”). FIG. 4B is a graph depicting plasma levels of NAMPT, IL-6, IL-8, and MIF in alive and dead ARDS patients, thus correlating plasma levels of these inflammatory cytokines with ARDS mortality. *** indicates p<0.001

FIGS. 5A-5B are graphical representations of plasma NAMPT level in healthy controls or pancreatitis patients. FIG. 5A is a graph depicting plasma NAMPT level in healthy controls or pancreatitis patients. FIG. 5B is a graph showing plasma NAMPT level in patients with mild, moderate or severe pancreatitis.

FIGS. 6A-6B are graphical representations of plasma NAMPT level in healthy controls or sepsis patients. FIG. 6A is a graph depicting plasma NAMPT level in healthy controls or sepsis patients. FIG. 6B is a graph showing plasma NAMPT level in sepsis patients with or without septic shock. *** indicates p<0.001

FIG. 7 is a graph depicting the correlation of ARDS mortality index with plasma NAMPT level, NAMPT SNPs and clinical covariate genotypes.

FIG. 8 graphically represents a correlation of NAMPT SNPs to risk of ARDS and ARDS mortality, as normalized over control. The left panel of FIG. 8 provides a graph depicting a correlation of single NAMPT SNP (odd ratio: 3.1) and two NAMPT SNP haplotype (odds ratio: 7.7) to risk of ARDS. The middle panel of FIG. 8 provides a graph depicting a correlation of single NAMPT SNP (odd ratio: 1.3) and two NAMPT SNP haplotype (odd ratio: 1.6) to ARDS mortality. The right panel of FIG. 8 provides a graph depicting a correlation of single NAMPT SNP (odd ratio: 4.3) to ARDS mortality. * p<0.05

FIGS. 9A-9B depict the effects of radiation on NAMPT expression, as evaluated in a mouse model of radiation-induced lung injury (RILI). FIG. 9A provides representative micrographs showing IHC staining for NAMPT in lung tissues of non-irradiated control mice (FIG. 9A, left panel) or irradiated RILI mice at 1 week (FIG. 9A, middle panel) or 4 weeks (FIG. 9A, right panel) post 20 Gy radiation exposure. FIG. 9B is a graphical depiction of NAMPT expression (% area) in lung tissue of irradiated mice at 1 week or 4 weeks post radiation exposure. Also depicted in the graph as negative control is NAMPT expression in lung tissue of non-irradiated mice.

FIGS. 10A-10C depict the effects of radiation on NAMPT expression in human tissues and blood. FIG. 10A provides representative micrographs showing IHC staining for NAMPT in human tonsillar epithelial tissue that was either non-irradiated (FIG. 10A, left panel) or exposed to 8 Gy ionizing radiation (IR) for 24 hours (FIG. 10A, right panel). FIG. 10B is a graph depicting plasma level of NAMPT in control subjects or in subjects undergoing radiotherapy for breast cancer or lung cancer. FIG. 10C is a graph depicting plasma level of NAMPT in control subjects or in patients with radiation pneumonitis. * indicates p<0.05

FIGS. 11A-11E depict lung inflammation (assessed by hematoxylin and eosin (H&E) staining), amount of bronchoalveolar lavage (B AL) protein and count of B AL-expressing cells in a mouse model of RILI. FIG. 11A provides representative micrographs showing H&E staining in lung tissues of non-irradiated control mice (inset of FIG. 11A, left panel) or irradiated RILI mice at 1 week (FIG. 11A, left panel) or 4 weeks (FIG. 11A, right panel) post radiation exposure. FIG. 11B is a graphical depiction of H&E staining (% area) in lung tissue of non-irradiated control mice or irradiated RILI mice at 1 week or 4 weeks post radiation exposure. FIG. 11C is a graphical representation of BAL protein levels (μg/ml) in lung tissues of non-irradiated control mice or irradiated RILI mice at 1 week or 4 weeks post radiation exposure. FIG. 11D is a graphical representation of BAL-expressing cells in lung tissues of non-irradiated control mice or irradiated RILI mice at 1 week or 4 weeks post radiation exposure. FIG. 11E is a graphical representation of RILI severity score of non-irradiated control mice or irradiated RILI mice at 1 week or 4 weeks post radiation exposure. * indicates p<0.05

FIGS. 12A-12E depict lung inflammation (assessed by H&E staining), amount of BAL protein and count of BAL-expressing cells in a mouse model of RILI that utilized wild-type (WT) and NAMPT heterozygous (Nampt^{+/-}) mice. FIG. 12A provides representative micrographs showing H&E staining in lung tissues of non-irradiated (control) WT mice (inset of FIG. 12A, left panel), irradiated (RILI) WT mice (FIG. 12A, left panel), or irradiated (RILI) Nampt^{+/-} mice (FIG. 12A, right panel) mice. FIG. 12B is a graphical depiction of H&E staining (% area) in lung tissue of non-irradiated (control) WT mice, non-irradiated (control) Nampt^{+/-} mice, irradiated (RILI) WT mice, or irradiated (RILI) Nampt^{+/-} mice. FIG. 12C is a graphical representation of BAL protein levels (μg/ml) in lung tissues of non-irradiated (control) WT mice, non-irradiated (control) Nampt^{+/-} mice, irradiated (RILI) WT mice, or irradiated (RILI) Nampt^{+/-} mice. FIG. 12D is a graphical representation of BAL-expressing cells in lung tissues of non-irradiated (control) WT mice, non-irradiated (control) Nampt^{+/-} mice, irradiated (RILI) WT mice, or irradiated (RILI) Nampt^{+/-} mice. FIG. 12E is a graphical representation of acute lung injury (ALI) severity score of non-irradiated (control) WT mice, non-irradiated (control) Nampt^{+/-} mice, irradiated (RILI) WT mice, or irradiated (RILI) Nampt^{+/-} mice.

FIGS. 13A-13E depict the effects of NAMPT-neutralizing antibodies on lung inflammation (assessed by H&E staining), amount of BAL protein and count of BAL-expressing cells, as evaluated in a murine model of RILI. FIG. 13A provides representative micrographs showing H&E staining in lung tissues of non-irradiated control mice (inset of FIG. 13A, left panel) or irradiated RILI mice that were injected with vehicle control (FIG. 13A, left panel), an anti-NAMPT polyclonal antibody (pAb) (FIG. 13A, middle panel), or an anti-NAMPT monoclonal antibody (mAb) (FIG. 13A, right panel) post radiation exposure. FIG. 13B is a graphical depiction of H&E staining (% area) in lung tissue of non-irradiated control mice or irradiated RILI mice that were injected with vehicle control, anti-NAMPT pAb, or anti-NAMPT mAb. FIG. 13C is a graphical representation of BAL protein levels (μg/ml) in lung tissues of non-irradiated control mice or irradiated RILI mice that were injected with vehicle control, anti-NAMPT pAb, or anti-NAMPT mAb. FIG. 13D is a graphical representation of number of BAL-expressing cells in lung tissues of non-irradiated control mice or irradiated RILI mice that were injected with vehicle control, anti-NAMPT pAb, or anti-NAMPT mAb. FIG. 13E is a graphical representation of ALI severity score of non-irradiated control mice or irradiated RILI mice that were injected with vehicle control, anti-NAMPT pAb, or anti-NAMPT mAb. * indicates p<0.05

FIGS. 14A-14D depict detection of NAMPT expression by ^{99m}Tc-labeled anti-NAMPT mAb probe. FIG. 14A provides representative autoradiograph images depicting detection of NAMPT expression by the ^{99m}Tc-labeled anti-NAMPT mAb probe in a non-irradiated control mouse (FIG. 14A, left panel) or in an irradiated (RILI) mouse exposed to 8 Gy partial body irradiation (PBI) (FIG. 14A, right panel). FIG. 14B provides representative autoradiograph images depicting detection of NAMPT expression by the ^{99m}Tc-labeled anti-NAMPT mAb probe in a non-irradiated control mouse (FIG. 14B, top panel) or in an irradiated (RILI) mouse (FIG. 14B, bottom panel). FIG. 14C is a graphical representation of ratio of lung activity over tissue background from left and right lungs of non-irradiated control mice or irradiated (RILI) mice. FIG. 14D is a graphical representation of radioactivity (% ID/g) in lung tissues of non-irradiated control mice or irradiated (RILI) mice. * indicates p<0.05

FIGS. 15A-15C depict the effects of a humanized anti-NAMPT mAb on BAL cell count, collagen deposition, and expression of lung tissue smooth muscle actin (SMA), as evaluated in a murine model of RILI 18 weeks after 20 Gy radiation exposure. FIG. 15A is a graph depicting number of BAL-expressing cells in lung tissues of irradiated RILI mice that were intraperitoneally injected with anti-NAMPT mAb or vehicle control. FIG. 15B provides representative images from western blot analyses showing expression of SMA in lung tissue homogenate of irradiated RILI mice that were intraperitoneally injected with anti-NAMPT mAb or vehicle control. FIG. 15C provides representative micrographs showing collagen deposition, as detected by Trichrome staining, in lung tissue of irradiated RILI mice that were intraperitoneally injected with anti-NAMPT mAb or vehicle control. * indicates p<0.05.

FIGS. 16A-16C depict the effects of a humanized anti-NAMPT mAb on inflammatory cell infiltration, edema and lung injury score, as evaluated in a rat model of trauma (blast)/ventilation-induced lung injury (VILI). FIG. 16A provides representative images and micrographs showing lung or lung tissue section of trauma/VILI challenged rats that were injected with vehicle control. The left panel of FIG. 16A provides representative image of lung from trauma/VILI challenged rats injected with vehicle control. The middle and right panels of FIG. 16A provides representative micrographs showing inflammatory cell infiltration and edema, as assessed by H&E staining, in trauma/VILI challenged rat injected with vehicle control. The inset of the rightmost panel of FIG. 16A provides representative micrograph showing H&E staining in lung tissue of rat not challenged with trauma/VILI. FIG. 16B provides representative images and micrographs showing lung or lung tissue section of trauma/VILI challenged rats that were injected with anti-NAMPT mAb. The left panel of FIG. 16B provides representative image of lung from trauma/VILI challenged rats injected with anti-NAMPT mAb. The middle and right panels of FIG. 16B provides representative micrographs showing inflammatory cell infiltration and edema, as assessed by H&E staining, in trauma/VILI challenged rats injected with anti-NAMPT mAb. FIG. 16C is a graph depicting lung injury score of trauma/VILI challenged rats that were injected with either anti-NAMPT mAb or vehicle control.

FIGS. 17A-17C depict the effects of NAMPT-neutralizing antibodies on inflammatory cell infiltration, edema and lung injury score, as evaluated in a murine LPS/VILI lung injury model. FIG. 17A provides a representative micrograph showing inflammatory cell infiltration and edema, as assessed by H&E staining, in LPS/VILI challenged mouse injected with vehicle control. The inset of FIG. 17A provides representative a micrograph showing H&E staining in lung tissue from mouse not challenged with LPS/VILI. FIG. 17B provides a representative micrograph showing inflammatory cell infiltration and edema, as assessed by H&E staining, in LPS/VILI challenged mouse injected with anti-NAMPT mAb. FIG. 17C is a graph depicting ALI severity score as assessed in LPS/VILI challenged mice that were injected with anti-NAMPT mAb, anti-NAMPT pAb or vehicle control (PBS). The graph in FIG. 17C also depicts ALI severity score of control mice that were not challenged with LPS/VILI. * indicates p<0.05; *** indicates p<0.001.

FIGS. 18A-18D depict detection of NAMPT expression by ^{99m}Tc-labeled anti-NAMPT mAb probe. FIG. 18A provides representative autoradiograph images depicting detection of NAMPT expression by the ^{99m}Tc-labeled anti-NAMPT mAb probe (PRONAMPTOR) (FIG. 18A, right panel) or a radiolabeled IgG control Ab (FIG. 18A, left panel) in mice that were exposed to 20 Gy total lung irradiation (WTLI). FIG. 18B provides representative autoradiograph images depicting detection of NAMPT expression by the ^{99m}Tc-labeled anti-NAMPT mAb probe in LPS challenged mouse 3 hours after LPS challenge (FIG. 18B, right panel) or in a non-challenged control mouse (FIG. 18B, left panel). FIG. 18C provides representative autoradiograph images depicting detection of NAMPT expression by the ^{99m}Tc-labeled anti-NAMPT mAb probe in lung of LPS challenged mouse 3 hours after LPS challenge (FIG. 18C, bottom panel) or in lung of a non-challenged control mouse (FIG. 18C, top panel). FIG. 18D is a graphical representation of uptake of the radiolabeled anti-NAMPT mAb probe, as assessed by radioactivity (% ID/g), in lung tissues of LPS challenged mouse at 3 hours and 18 hours post LPS challenge or in lung tissues of non-challenged control mice. * indicates p<0.05.

FIG. 19 provides representative micrographs showing IHC staining for NAMPT in lung tissues of idiopathic pulmonary fibrosis (IPF) patients. Arrows indicate NAMPT expression in fibroblasts within fibrotic regions of IPF lung tissue. The top panel provides micrographs in 4× magnification, and the bottom panel provides micrographs in 40× magnification.

FIGS. 20A-20C depict NAMPT expression in plasma and lung tissues of IPF patients. FIG. 20A is a graphical representation of NAMPT level in plasma samples from IPF patients or healthy controls. FIG. 20B is a graphical representation of NAMPT level in plasma samples from dead IPF patients, alive IPF patients, treated IPF patients or untreated IPF patients. FIG. 20C is a graphical representation of Nampt mRNA levels in fibroblasts isolated from advanced stage IPF patients or early stage IPF patients. * indicates p<0.05

FIG. 21 is a graphical representation of soluble collagen (μg/lung), indicative of fibrosis, in whole lung of bleomycin-challenged WT mice or bleomycin-challenged Nampt^{+/-} mice. The graph also shows whole lung soluble collagen of control WT mice or control Nampt^{+/-} mice that were not challenged with bleomycin. * indicates p<0.05

FIGS. 22A-22D depict NAMPT expression and NAMPT SNPs in pulmonary artery hypertension (PAH) patients. FIG. 22A provides representative micrographs showing IHC staining for NAMPT in lung tissues from idiopathic pulmonary artery hypertension (IPAH) patients. The inset of FIG. 22A provides a representative micrograph showing IHC staining for NAMPT in lung tissue from healthy control. FIG. 22B is a graphical representation of NAMPT level in plasma samples obtained from patients with PAH, patients with non-PAH lung diseases, or healthy control subjects. FIG. 22C provides representative images from western blot analyses showing expression of NAMPT in lung tissue from IPAH patients or healthy control subjects (“Nor”). FIG. 22D is a graphical representation of correlation of NAMPT promoter SNP to right ventricular (RV) indices.

FIGS. 23A-23B depict effects of a humanized anti-NAMPT mAb on right ventricular systolic pressure (RVSP) and pulmonary artery thickness, as evaluated in a rat monocrotaline (MCT) model of PAH. FIG. 23A is a graphical representation of RVSP in MCT-challenged rats that were injected with either anti-NAMPT mAb or vehicle control (control MCT mice). FIG. 23B provides representative micrographs showing pulmonary artery thickness, as assessed by H&E staining, in MCT-challenged rats that were injected with either anti-NAMPT mAb (FIG. 23B, right panel) or vehicle control (FIG. 23B, left panel). * indicates p<0.05.

### DETAILED DESCRIPTION OF THE INVENTION

### Definitions

In order that the invention may be more readily understood, certain terms are first defined. In addition, it should be noted that whenever a value or range of values of a parameter are recited, it is intended that values and ranges intermediate to the recited values are also part of this invention. It is also to be noted that as used herein, the singular forms “a,” “and” and “the” include plural references unless the context clearly dictates otherwise.

The term “single nucleotide polymorphism,” or “SNP,” as used interchangeably here, refers to a DNA sequence variation occurring when a single nucleotide in the genome (or other shared sequence) differs between members of a species (or between paired chromosomes in an individual). A SNP can occur in either a coding or non-coding region of the genome of an organism.

The term “NAMPT” or “eNAMPT”, used interchangeably herein, refers to the secreted form of nicotinamide phosphoribosyltransferase (NAMPT) unless specifically mentioned to relate to a non-secreted form (e.g., intracellular NAMPT or NAMPT nucleic acids). The amino acid sequence of secreted human NAMPT (also referred to as human eNAMPT) is provided below as SEQ ID NO: 1 (see also NCBI Gene Ref. No. NC_000007.14 and Protein Ref. No. NP_005737.1).

(SEQ ID NO: 1)
MNPAAEAEFN ILLATDSYKV THYKQYPPNT SKVYSYFECR
EKKTENSKLR KVKYEETVFY GLQYILNKYL KGKVVTKEKI
QEAKDVYKEH FQDDVFNEKG WNYILEKYDG HLPIEIKAVP
EGFVIPRGNV LFTVENTDPE CYWLTNWIET ILVQSWYPIT
VATNSREQKK ILAKYLLETS GNLDGLEYKL HDFGYRGVSS
QETAGIGASA HLVNFKGTDT VAGLALIKKY YGTKDPVPGY
SVPAAEHSTI TAWGKDHEKD AFEHIVTQFS SVPVSVVSDS
YDIYNACEKI WGEDLRHLIV SRSTQAPLII RPDSGNPLDT
VLKVLEILGK KFPVTENSKG YKLLPPYLRV IQGDGVDINT
LQEIVEGMKQ KMWSIENIAF GSGGGLLQKL TRDLLNCSFK
CSYVVTNGLG INVFKDPVAD PNKRSKKGRL SLHRTPAGNF
VTLEEGKGDL EEYGQDLLHT VFKNGKVTKS YSFDEIRKNA
QLNIELEAAHH

NAMPT is also referred to as pre-B cell colony enhancing factor (PBEF) or visfatin.

The term “baseline”, as used herein, refers to a reference or control measurement, e.g., a control level of NAMPT expression from a healthy subject (i.e., a subject not having prostate cancer) or a subject having indolent prostate cancer.

As used herein, a “NAMPT inhibitor” or an “inhibitor of NAMPT” refers to an agent that reduces or prevents NAMPT activity. In some embodiments, a NAMPT inhibitor binds to NAMPT, resulting in inhibition of the biological activity of NAMPT.

As used herein, the terms “NAMPT antibody” or “anti-NAMPT antibody” or “anti-eNAMPT antibody,” used interchangeably herein, refer to an antibody that specifically binds to the secreted form of NAMPT (also referred to herein as eNAMPT). In a preferred embodiment, the antibody specifically binds to human NAMPT (hNAMPT). Preferably, NAMPT antibodies inhibit the biological activity of NAMPT. It will be appreciated that modified NAMPT activity may be measured directly using art recognized techniques or may be measured by the impact the altered activity has downstream.

The term “aggressive prostate cancer”, as used herein, refers to prostate cancer that is defined as having a Gleason severity score of score of 7 to 10 or a metastatic prostate cancer.

The term “indolent prostate cancer” refers to a low grade prostate cancer having a Gleason severity score of score of 6 or less.

The term “level” or “amount” as used herein refers to the measurable quantity of a biomarker, e.g., a level of NAMPT expression. The amount may be either (a) an absolute amount as measured in molecules, moles or weight per unit volume or cells or (b) a relative amount, e.g., measured by densitometric analysis.

The term “sample” as used herein refers to material (e.g., a collection of similar cells or tissue) obtained from a subject. The sample may be solid tissue as from a fresh, frozen and/or preserved organ or tissue sample or biopsy or aspirate; blood or any blood constituents; or bodily fluids, such as blood, serum, plasma, urine, saliva, sweat or synovial fluid. In some embodiments, the synovitis biomarker is obtained from a serum sample. In some embodiments, the cartilage degradation biomarker is obtained from a urine sample.

The terms “patient,” “individual,” or “subject” are used interchangeably herein, and refer to a mammal, particularly, a human. The patient may have no disease, mild, intermediate or severe disease. The patient may be treatment naïve, responding to any form of treatment, or refractory. The patient may be an individual in need of treatment or in need of diagnosis based on particular symptoms or family history. In some embodiments, a subject is a human subject that has been diagnosed with, previously treated for, or has symptoms of, non-aggressive or indolent prostate cancer. In other embodiments, a subject is a healthy human subject that has not been diagnosed with, not previously treated for, or does not have symptoms of, prostate cancer. In yet another embodiment, a subject is a human subject that has been diagnosed with, previously treated for, or has symptoms of, aggressive prostate cancer.

Terms such as “treating” or “treatment” or “to treat” or “alleviating” or “to alleviate” refer to therapeutic measures that cure, slow down, lessen symptoms of, and/or halt or slow the progression of an existing diagnosed pathologic condition or disorder.

Terms such as “prevent,” and the like refer to prophylactic or preventative measures that prevent the development of a targeted pathologic condition or disorder.

The term “effective amount” or “therapeutically effective amount” are used interchangeably herein, and refer to an amount of an agent that is effective to achieve a particular biological result. Such results may include, but are not limited to, the inhibition of NAMPT expression or activity, or the expression or activity of signaling molecules which are downstream of NAMPT as determined by any means suitable in the art.

### Diseases/Conditions

Provided are methods of determining risk of developing conditions associated with NAMPT expression. Also provided are methods of diagnosing and/or treating such conditions. In some embodiments, the NAMPT-associated condition is an inflammatory condition, such as acute respiratory distress syndrome (ARDS), radiation-induced lung injury (RILI), pulmonary hypertension, or pulmonary fibrosis. In some embodiments, the NAMPT-associated condition is prostate cancer.

ARDS is a respiratory disorder characterized by widespread inflammation in the lungs. Symptoms include one or more of shortness of breath, rapid breathing, bluish skin coloration, low blood pressure, confusion, and extreme tiredness. Without being bound by theory, it is believed that ARDS is caused by fluid leaking from blood vessels in the lungs into air sacs where blood is oxygenated (normally, a protective membrane keeps this fluid in the vessels). It is further believed that such leaking can be caused by one or more of sepsis; inhalation of harmful substances (e.g., smoke, chemical fumes, near-drowning, aspirating vomit); severe pneumonia; head, chest, or other major injury (e.g., falls or car crashes that damage the lungs); coronavirus disease 2019 (COVID-19); pancreatitis; blood transfusions; and burns. Adverse outcomes associated with ARDS include blood clots; collapsed lungs (pneumothorax); infections; scarring (pulmonary fibrosis); long-term breathing problems (i.e., last more than 2 months, more than two years, or lifelong); depression; problems with memory or thinking clearly; tiredness; and muscle weakness. ARDS is discussed in greater detail in Matthay et al., “Acute respiratory distress syndrome,” Nature Reviews Disease Primers, 5(18): 1-22 (2019), which is incorporated herein by reference in its entirety.

RILI is characterized by damage to the lungs as a result of exposure to ionizing radiation. Symptoms include one or more of dyspnea, cough, fever, and chest pain. Without being bound by theory, it is believed that RILI is caused by one of two primary mechanisms: direct DNA damage, or generation of reactive oxygen species. Adverse outcomes associated with RILI include pneumonitis, tissue fibrosis, necrosis, atrophy, and vascular injury. RILI is discussed in greater detail in Giuranno et al., “Radiation-Induced Lung Injury (RILI),” Frontiers in Oncology, 9 (Article 877): 1-16 (2019), which is incorporated herein by reference in its entirety.

Pulmonary hypertension is a type of high blood pressure that affects arteries in the lungs and/or right side of the heart. In one form of pulmonary hypertension—pulmonary arterial hypertension—blood vessels in the lungs are narrowed, blocked, or destroyed. Symptoms of pulmonary hypertension include shortness of breath (dyspnea); fatigue; dizziness or fainting spells (syncope); chest pressure or pain; swelling (edema) in ankles, legs, and/or abdomen (ascites); bluish color in lips and/or skin (cyanosis); and/or racing pulse or heart palpitations. Without being bound by theory, it is believed that pulmonary hypertension is caused genetic mutations; use of diet pills or illegal drugs such as methamphetamines; heart problems (e.g., congenital heart disease); connective tissue disorders (e.g., scleroderma, lupus, etc); HIV infection; chronic liver disease (cirrhosis); left-sided heart valve disease; failure of lower left heart chamber; chronic obstructive pulmonary disease; pulmonary fibrosis; obstructive sleep apnea; long-term exposure to high altitudes; blood disorders (e.g., polycythemia; essential thrombocythemia); inflammatory disorders (e.g., sarcoidosis; vasculitis); metabolic disorders (e.g., glycogen storage disease); kidney disease; and tumors pressing against pulmonary arteries. Adverse outcomes associated with pulmonary hypertension include heart enlargement; heart failure; blood clots; arrhythmia; bleeding in lungs; and pregnancy complications. Pulmonary hypertension is discussed in greater detail in Hambly et al., “Pulmonary hypertension: diagnostic approach and optimal management,” CMAJ, 188(11): 804-812 (2016), which is incorporated herein by reference in its entirety.

Pulmonary fibrosis is a lung disease that occurs when lung tissue becomes damaged and scarred. Pulmonary fibrosis is characterized by a thickening of tissue around and between alveoli in the lungs, making it difficult to pass oxygen into the bloodstream. Symptoms of pulmonary fibrosis include dyspnea, dry cough, fatigue, unexplained weight loss, aching muscles and joints, and widening and rounding of the tips of the fingers or toes (clubbing). Without being bound by theory, it is believed that pulmonary fibrosis is caused by occupational and environmental factors (e.g., exposure to silica dust, asbestos fibers, hard metal dusts, coal dusts, grain dusts, or bird/animal droppings); radiation treatments (e.g., radiation therapy for lung or breast cancer); medications (e.g., chemotherapy drugs such as methotrexate or cyclophosphamide; heart medications such as amiodarone; and/or antibiotics such as nitrofurantoin or ethambutol); anti-inflammatory drugs such as rituximab or sulfasalazine); and/or medical conditions (e.g., dermatomyositis; polymyositis; mixed connective tissue disease; systemic lupus erythematosus; rheumatoid arthritis; sarcoidosis; scleroderma; and/or pneumonia). Adverse outcomes associated with pulmonary fibrosis include pulmonary hypertension; cor pulmonale; respiratory failure; lung cancer; blood clots in lungs; lung infections; and collapsed lungs. Pulmonary fibrosis is discussed in greater detail in Baratt et al., “Idiopathic Pulmonary Fibrosis (IPF): An Overview,” J. Clin. Med., 7(8): 1-21 (2018), which is incorporated herein by reference in its entirety.

Coronavirus disease 2019 (COVID-19) is a severe acute respiratory syndrome caused by coronavirus 2 (SARS-CoV-2). SARS-CoV-2 has a diameter of 60 nm to 140 nm and distinctive spikes, ranging from 9 nm to 12 nm, giving the virions the appearance of a solar corona. Through genetic recombination and variation, coronaviruses can adapt to and infect new hosts. SARS-CoV-2 infection may be asymptomatic or it may cause a wide spectrum of symptoms. Exemplary symptoms include fever, cough, shortness of breath, weakness, fatigue, nausea, vomiting, and changes to taste and smell. Adverse outcomes include diffuse intravascular coagulation; inflamed lung tissues and pulmonary endothelial cells; deep venous thrombosis; pulmonary embolism; thrombotic arterial complications (e.g., limb ischemia; ischemic stroke; myocardial infarction); sepsis; and multi-organ failure. SARS-CoV-2 infection is discussed in greater detail in Wiersinga et al., “Pathophysiology, Transmission, Diagnosis, and Treatment of Coronavirus Disease 2019 (COVID-2019): A Review,” JAMA, doi:10.1001/jama.2020.12839 (published online Jul. 10, 2020), which is incorporated herein by reference in its entirety.

Prostate cancer is a cancer that occurs in the prostate. Symptoms of prostate cancer include difficulty urinating, decreased force in the stream of urine, blood in semen, discomfort in pelvic area, bone pain, and erectile dysfunction. Without being bound my theory, it is believed that prostate cancer is caused by mutations in abnormal cells' DNA that causes the cells to grow and divide more rapidly than the normal cells, with abnormal cells accumulating and forming a tumor that can invade nearby tissue and/or metastasize to other parts of the body. Adverse consequences of prostate cancer include metastasis to other organs or bones (e.g., through bloodstream or lymphatic system); incontinence; and erectile dysfunction. Prostate cancer is discussed in greater detail in Litwin et al., “The Diagnosis and Treatment of Prostate Cancer: A Review,” JAMA, 317(24): 2532-2542 (2017), which is incorporated herein by reference in its entirety.

### Biomarkers, Single Nucleotide Polymorphisms, and Uses Thereof

Provided are methods of determining risk of developing conditions associated with NAMPT expression. Also provided are methods of diagnosing such conditions. Some embodiments comprise determining progression of a NAMPT-associated condition. Some embodiments comprise determining efficacy of treatment of a NAMPT-associated condition.

In some embodiments, the NAMPT-associated condition is an inflammatory condition, such as acute respiratory distress syndrome (ARDS), radiation-induced lung injury (RILI), pulmonary hypertension, or pulmonary fibrosis. In some embodiments, the NAMPT-associated condition is prostate cancer. In some embodiments, the subject has indolent prostate cancer and may be at risk for developing a more aggressive form of prostate cancer.

Some embodiments comprise detecting a presence or absence of NAMPT in a sample. Some embodiments comprise detecting a level of NAMPT in a sample. In some embodiments, a subject is determined to have, or be at risk of developing, a condition based on the presence or level (e.g., increased level) of NAMPT in the sample. In some embodiments, the subject is determined not to have, or not to be at risk of developing, a condition based on the absence of or a low or decreased level of NAMPT in the sample.

Some embodiments comprise detecting a presence, absence, or level of one or more additional biomarkers, such as cytokine chemokines (e.g., IL-6, IL-8, IL-1b, and/or IL-RA); dual functioning enzymes such as macrophage migration inhibitory factor); vascular injury markers (e.g., VEGRA, S1PR3, and/or angiopoietin 2); and/or advanced glycosylation end product pathway markers (e.g., HMGB1 and/or soluble RAGE). Some embodiments comprise determining an increased or decreased risk that a subject has or will develop a condition based on the level (e.g., an elevated level or a decreased level) of one or more of the preceding markers (e.g., in combination with the presence, absence, or level of NAMPT).

Single nucleotide polymorphisms (SNPs) are located in gene promoters, exons, introns as well as 5′- and 3′-untranslated regions (UTRs) and affect gene expression by different mechanisms. Provided are SNPs located in the promoter region of the human NAMPT gene.

In some embodiments, one or more of the following SNPs are associated with the inflammatory condition or prostate cancer (e.g., aggressive prostate cancer): rs7789066 (position: chr7:106287306 (GRCh38.p12)); rs116647506 (position: chr7:106287180 (GRCh38.p12)); rs61330082 (position: chr7:106286419 (GRCh38.p12)); rs114382471 (position: chr7:106286288 (GRCh38.p12)); rs9770242 (position: chr7:106285885 (GRCh38.p12)); rs59744560 (position: chr7:106285832 (GRCh38.p12)); rs190893183 (position: chr7:106285663 (GRCh38.p12)); and rs1319501 (position: chr7:106285307 (GRCh38.p12)). These SNPs, and their presence in subjects with various diseases, are shown in Table 1.

TABLE 1
NAMPT Promoter SNPs identified for ARDS and
prostate cancer risk.

| | | MAF | | |
|---|---|---|---|---|
| Disease | SNP | Global | AD | ED |
| ARDS | rs7789066 (−2422) | 0.06 | 0.14* | 0.07 |
| Cancer | rs116647506 (−2296) | 0.03 | 0.06* | 0.02 |
| ARDS; | rs61330082 (−1535) | 0.27 | 0.07* | 0.30 |
| Cancer | | | | |
| Cancer | rs114382471 (−1404) | 0.01 | 0.05* | 0 |
| ARDS | rs9770242 (−1001) | 0.13 | 0.21 | 0.20 |
| ARDS | rs59744560 (−948) | 0.05 | 0.01 | 0.12 |
| Cancer | rs190893183 (−779) | 0.003 | 0.01* | 0 |
| Unknown | rs1319501 (−423) | 0.16 | 0.30 | 0.20 |

*denotes SNPs that are over- or under-represented in African descent individuals.

Some embodiments comprise detecting 2, 3, 4, 5, 6, 7, or 8 SNPs selected from the group consisting of rs7789066; rs116647506; rs61330082; rs114382471; rs9770242; rs59744560; rs190893183; and rs1319501.

In some embodiments, a SNP used in the methods described herein is rs7789066, rs61330082, rs9770242, and/or rs59744560. In some embodiments, a SNP used in the methods described herein is rs116647506, rs114382471, rs190893183, and/or rs1319501.

Without being bound by theory, it is believed the SNPs described herein may contribute to dysregulation of cellular processes including dysregulation of inflammatory signaling pathways (e.g., NFkB-dependent inflammatory cascades) and lead to the progression or metastasis of cells, resulting in inflammatory conditions and/or cancer (e.g., prostate cancer). It is contemplated that SNPs that occur within the promoter region of human NAMPT cause increased NAMPT promoter activity. The increased activity leads to an increased expression of NAMPT and subsequently, increased plasma levels of NAMPT. The possible increase in the levels of NAMPT activate the evolutionarily-conserved, NFkB-dependent inflammatory cascades via Toll-like receptor 4 (TLR4). The enhanced production of cytokines in turn enhance the transition to an inflammatory phenotype or invasive prostate cancer phenotype, thus increasing the risk of a subject developing the inflammatory condition or prostate cancer. NAMPT also participates in tumor/host cross-talk to influence the microenvironment and prostate cancer invasion and metastasis.

Provided are methods for identifying a subject at risk of developing an inflammatory condition (e.g. ARDS, RILI, pulmonary hypertension, or pulmonary fibrosis) or aggressive prostate cancer. In some embodiments, identification of such a subject includes obtaining a sample from a subject who may be at risk for developing the inflammatory condition or aggressive prostate cancer, and subsequently testing for the presence or absence of at least one of the foregoing SNPs in the sample. In some embodiments, the presence of at least one SNP in the sample indicates that the subject is at risk for developing the inflammatory condition. In some embodiments, the presence of at least one SNP in the sample indicates that the subject is at risk for developing aggressive prostate cancer.

An example of a subject who may be at risk for developing aggressive prostate cancer is a subject having indolent prostate cancer. Thus, identification of a SNP described herein in a patient having indolent prostate cancer can be used to predict whether the patient is susceptible or at risk for developing aggressive prostate cancer. In some embodiments, the presence of 2, 3, 4, 5, 6, 7, or 8 SNPs selected from the group consisting of rs7789066; rs116647506; rs61330082; rs114382471; rs9770242; rs59744560; rs190893183; and rs1319501 indicates the subject has or is at risk of developing prostate cancer. Some embodiments comprise diagnosing a subject as having prostate cancer based on the presence of one or more SNPs.

In some embodiments, the indolent prostate cancer is sporadic or inherited. In the inherited form of prostate cancer, subjects of African or European descent are at an increased risk of developing prostate cancer.

In some embodiments, detection of at least one SNP in the promoter element of NAMPT from a sample can be achieved by SNP genotyping. Generally, SNP genotyping includes steps of, for example, collecting a biological sample from a test subject (e.g., sample of biopsied tissues, cells, fluids, secretions, etc.), isolating nucleic acids (e.g., genomic DNA, mRNA or both) from the cells of the sample, contacting the nucleic acids with one or more primers which specifically hybridize to a region of the isolated nucleic acid containing a target SNP under conditions such that hybridization and amplification of the target nucleic acid region occurs, and determining the nucleotide present at the SNP position of interest, or, in some assays, detecting the presence or absence of an amplification product (assays can be designed so that hybridization and/or amplification will only occur if a particular SNP allele is present or absent). SNP genotyping can identify SNPS that are either homozygous or heterozygous. In some embodiments of the method described herein, the at least one SNP is homozygous. In other embodiments, the at least one SNP is heterozygous.

Other methods of detecting SNPs are known to the art and can be applied to the present methods. For example, an assay system that is commercially available and can be used to identify a nucleotide occurrence of one or more SNPs is the SNP-IT™ assay system (Orchid BioSciences, Inc.; Princeton N.J.). In general, the SNP-IT™ method is a three step primer extension reaction. In the first step a target nucleic acid molecule is isolated from a sample by hybridization to a capture primer, which provides a first level of specificity. In a second step the capture primer is extended from a terminating nucleotide triphosphate at the target SNP site, which provides a second level of specificity. In a third step, the extended nucleotide triphosphate can be detected using a variety of known formats, including, for example, by direct fluorescence, indirect fluorescence, an indirect colorimetric assay, mass spectrometry, or fluorescence polarization. Reactions conveniently can be processed in 384 well format in an automated format using a SNPSTREAM™ instrument (Orchid BioSciences, Inc.).

Nucleic acid samples from a sample taken from a subject can be genotyped to determine the presence and identity of a SNP of interest by methods known to a person of skill in the art. The neighboring sequence can be used to design SNP detection reagents such as oligonucleotide probes, which may optionally be implemented in a kit format. Exemplary SNP genotyping methods are described in Chen et al., “Single nucleotide polymorphism genotyping: biochemistry, protocol, cost and throughput”, Pharmacogenomics J. 2003; 3(2):77-96; Kwok et al., “Detection of single nucleotide polymorphisms”, Curr Issues Mol. Biol. 2003 April; 5(2):43-60; Shi, “Technologies for individual genotyping: detection of genetic polymorphisms in drug targets and disease genes”, Am J Pharmacogenomics. 2002; 2(3): 197-205; and Kwok, “Methods for genotyping single nucleotide polymorphisms”, Annu Rev Genomics Hum Genet 2001; 2:235-58. Exemplary techniques for high-throughput SNP genotyping are described in Marnellos, “High-throughput SNP analysis for genetic association studies”, Curr Opin Drug Discov Devel. 2003 May; 6(3):317-21. Common SNP genotyping methods include, but are not limited to, TaqMan assays, molecular beacon assays, nucleic acid arrays, allele-specific primer extension, allele-specific PCR, arrayed primer extension, homogeneous primer extension assays, primer extension with detection by mass spectrometry, pyrosequencing, multiplex primer extension sorted on genetic arrays, ligation with rolling circle amplification, homogeneous ligation, OLA (U.S. Pat. No. 4,988,167), multiplex ligation reaction sorted on genetic arrays, restriction-fragment length polymorphism, single base extension-tag assays, and the Invader assay. Such methods may be used in combination with detection mechanisms such as, for example, luminescence or chemiluminescence detection, fluorescence detection, time-resolved fluorescence detection, fluorescence resonance energy transfer, fluorescence polarization, mass spectrometry, and electrical detection. Various methods for detecting polymorphisms include, but are not limited to, methods in which protection from cleavage agents is used to detect mismatched bases in RNA/RNA or RNA/DNA duplexes (Myers et al, Science 230: 1242 (1985); Cotton et al, PNAS 85:4397 (1988); and Saleeba et al., Meth. Enzymol. 217:286-295 (1992)), comparison of the electrophoretic mobility of variant and wild type nucleic acid molecules (Orita et al., PNAS 86:2766 (1989); Cotton et al., Mutat. Res. 285: 125-144 (1993); and Hayashi et al., Genet. Anal. Tech. Appl. 9:73-79 (1992)), and assaying the movement of polymorphic or wild-type fragments in polyacrylamide gels containing a gradient of denaturant using denaturing gradient gel electrophoresis (DGGE) (Myers et al., Nature 313:495 (1985)). Sequence variations at specific locations can also be assessed by nuclease protection assays such as RNase and SI protection or chemical cleavage methods.

In some embodiments, detecting a SNP in the NAMPT promoter sequence comprises contacting a sample from a subject with an oligonucleotide probe that selectively hybridizes to a nucleotide sequence comprising the SNP, or a nucleotide sequence complementary thereto, and detecting selective hybridization of the oligonucleotide probe. In certain embodiments, an oligonucleotide probe that selectively hybridizes to a nucleotide sequence comprising a SNP includes 100-500 (e.g., 100, 125, 150, 175, 200, 225, 250, 275, 300, 325, 350, 375, 400, 425, 450, 475, or 500) base pairs on each side surrounding the SNP. For example, an oligonucleotide probe that selectively hybridizes to a nucleotide sequence comprising a SNP can include 200 base pairs on each side surrounding the SNP. In particular embodiments, an oligonucleotide probe comprising the nucleotide sequence set forth in SEQ ID NO: 18 selectively hybridizes to a nucleotide sequence comprising rs7789066; an oligonucleotide probe comprising the nucleotide sequence set forth in SEQ ID NO: 19 selectively hybridizes to a nucleotide sequence comprising rs61330082; an oligonucleotide probe comprising the nucleotide sequence set forth in SEQ ID NO: 20 selectively hybridizes to a nucleotide sequence comprising rs9770242; an oligonucleotide probe comprising the nucleotide sequence set forth in SEQ ID NO: 21 selectively hybridizes to a nucleotide sequence comprising rs59744560; and/or an oligonucleotide probe comprising the nucleotide sequence set forth in SEQ ID NO: 22 selectively hybridizes to a nucleotide sequence comprising rs1319501. Exemplary oligonucleotide probes that can selectively hybridize to nucleotide sequences comprising the SNPs are provided in Table 2 below:

TABLE 2
Exemplary oligonucleotide probes for detecting SNPs

| | | Sequence |
|---|---|---|
| Description | Sequence | identifier |
| probe for | AATGTGGGCTTTGTTTATGGTAGTATTTTTTTAAGAT | SEQ ID |
| detecting | GCAAAATTTGATCTTGCAATCTTTGAGTTGAATTTG | NO: 18 |
| rs7789066 | CAGTTTTAAAATAAAAAGGTCTTATATCTGTGCAAA | |
| (SNP | GAAAAAATATTGTATTGACATTGCTTGTTAAATTAA | |
| underlined) | GGAGTGAGGCCTGCACAAGTATTAGTAATGTGAAT | |
| | CCTCACAGTAGTCTCCAGAGAAAAAAAATGACAAT | |
| | GAAGTCATGTTACCAATAGGACAATCACCATTTGCC | |
| | TGAGATAGAAATAGGCACATTCTCTATGTAACTACA | |
| | TGCTTAAGCTGGAGCAATTCAGAATTAATTGGGGTT | |
| | TAGAACTATGAAATTATCACTGAAAACAGAGCCAA | |
| | GATTTCATTTTAAAATGGCCTCCCCTGAAAGACAGT | |
| | TTAACAG | |
| probe for | AGTGGAACTTGTGAATTGAGATTCATAGTGGAACTT | SEQ ID |
| detecting | GTGAATTGAGATTCATCTCGAAACTGGAGGCATGG | NO: 19 |
| rs61330082 | CTGAGACTTCTAATAAAGACAACCTCAGTCAACACT | |
| (SNP | ATGTCTTGAAGTCAGTATATATTTTTGACAATCACC | |
| underlined) | TCATCTACACGTAGATACAATACAGGGCAAAGATC | |
| | ATGGAAGTGGAAGGTATCACCAGGCACTCACCAAT | |
| | GTAGTAAATACTAGTACACTTACAATTATTTTCAGC | |
| | AACGAGGTTTGAAACAAGAGGGCTTATGTATTTATT | |
| | GGTTGATCTTCCCTGTGTTTTACCGGGGAAAATTAT | |
| | TTGTAAACGCATTTAAACAAATTATTATTTCTATTTT | |
| | GAGACGGAGTCTCGCTCTGTCGCCCAGGCTGGAGT | |
| | GCAGTGGC | |
| probe for | CCGCTTTCCTCCGGCGGCTCTGTCTATGGCTGAGCT | SEQ ID |
| detecting | CTTTGATCCTTTGAGAGATGGTTTGACTTTTCCCGA | NO: 20 |
| rs9770242 | GCAAAGAGCCTGCGTTGAAAAGCGGGGGTGGAATT | |
| (SNP | CAGTCCTCACAGATAATGAGGGGACAAGACCTAAT | |
| underlined) | TGAACCGAGTATTGCCGGGAAGGAAAAGGCAACGG | |
| | GCCAAGCCTTTGACAGGGTGCGACACTGACTTTTAT | |
| | CATCGTTATAGTCTTTAAATCCTGGGAAACGAGTTG | |
| | GCAACCCCAAAATAAAGAAGTGTAATGACGTCTGA | |
| | TGACTTCACCCAAATACAGACCATTCCAAGAAAGA | |
| | CTTGCGCAGTTCTCATGCGTGGTTGCGTTTTTGCAT | |
| | AAAACTAAGATTCCCTTTGTCCGCATGTTTAATAGC | |
| | TTAAAAATAA | |
| probe for | GAGCTGCGGTGAGGAGTGAGGCTGAGGGGCCCCTT | SEQ ID |
| detecting | TCATCTGATGCAGCGACTCCGCTTTCCTCCGGCGGC | NO: 21 |
| rs59744560 | TCTGTCTATGGCTGAGCTCTTTGATCCTTTGAGAGA | |
| (SNP | TGGTTTGACTTTTCCCGAGCAAAGAGCCTGCGTTGA | |
| underlined) | AAAGCGGGGGTGGAATTCAGTCCTCACAGATAATG | |
| | AGGGGACAAGACCTAATTGAACCGAGTATTGCCGG | |
| | GAAGGAAAAGGCAACGGGCCAAGCCTTTGACAGGG | |
| | TGCGACACTGACTTTTATCATCGTTATAGTCTTTAA | |
| | ATCCTGGGAAACGAGTTGGCAACCCCAAAATAAAG | |
| | AAGTGTAATGACGTCTGATGACTTCACCCAAATACA | |
| | GACCATTCCAAGAAAGACTTGCGCAGTTCTCATGCG | |
| | TGGTTGCGTT | |
| probe for | GGGAGCTCTGGCGGACTCCCCACCTCGGTTCCCCCG | SEQ ID |
| detecting | CCTTCACCCCGTCACCCTCCGGGGGCCGAGAAAGG | NO: 22 |
| rs1319501 | GCGGGGCGCGGCAGCGCGCTGCGCAGTGCGCGGAG | |
| (SNP | GCGGGGCGGGGAGGAGGACGTGATGCACGCGCTCT | |
| underlined) | TCCTCCCAGACGCCAGCTCTGGGAAGCTGGAGGCA | |
| | GCGGGGCAGCCCCGGCGCGTGACCCGGGCGCTTAC | |
| | CTAAGTTCGAGTTCCCGGCACGGGCGCGGGAGGGC | |
| | GGGGCCTGGAGGGGGCGTTCCCAGCTTTGCCAGTG | |
| | CCACGAGGAGCCGGTTCGCCCGCCCCGCCTGGGAC | |
| | CTTCCGTCCTACCCAGTCCTGGCCGGTTTTCTGGGT | |
| | CCTCCTGAAGTCACGCCACCCGGCTAGGGGGCGAG | |
| | GAGCCTCCTACTGC | |

In some embodiments, SNP genotyping is performed using the TaqMan assay, which is also known as the 5′ nuclease assay (U.S. Pat. Nos. 5,210,015 and 5,538,848). The TaqMan assay detects the accumulation of a specific amplified product during PCR. The TaqMan assay utilizes an oligonucleotide probe labeled with a fluorescent reporter dye and a quencher dye. The reporter dye is excited by irradiation at an appropriate wavelength, it transfers energy to the quencher dye in the same probe via a process called fluorescence resonance energy transfer (FRET). When attached to the probe, the excited reporter dye does not emit a signal. The proximity of the quencher dye to the reporter dye in the intact probe maintains a reduced fluorescence for the reporter. The reporter dye and quencher dye may be at the 5′ most and the 3′ most ends, respectively, or vice versa. Alternatively, the reporter dye may be at the 5′ or 3′ most end while the quencher dye is attached to an internal nucleotide, or vice versa. In yet another embodiment, both the reporter and the quencher may be attached to internal nucleotides at a distance from each other such that fluorescence of the reporter is reduced. During PCR, the 5′ nuclease activity of DNA polymerase cleaves the probe, thereby separating the reporter dye and the quencher dye and resulting in increased fluorescence of the reporter. Accumulation of PCR product is detected directly by monitoring the increase in fluorescence of the reporter dye. The DNA polymerase cleaves the probe between the reporter dye and the quencher dye only if the probe hybridizes to the target SNP-containing template which is amplified during PCR, and the probe is designed to hybridize to the target SNP site only if a particular SNP allele is present. In some embodiments of the method, the oligonucleotide comprises a bilabeled oligonucleotide probe, comprising a fluorescent moiety and a fluorescent quencher.

Preferred TaqMan primer and probe sequences can readily be determined using the SNP and associated nucleic acid sequence information provided herein. A number of computer programs, such as Primer Express (Applied Biosystems, Foster City, Calif.), can be used to rapidly obtain optimal primer/probe sets. It will be apparent to one of skill in the art that such primers and probes for detecting the SNPs of the present invention are useful in prognostic assays for a variety of disorders including cancer, specifically, prostate cancer, and can be readily incorporated into a kit format. The present invention also includes modifications of the Taqman assay well known in the art such as the use of Molecular Beacon probes (U.S. Pat. Nos. 5,118,801 and 5,312,728) and other variant formats (U.S. Pat. Nos. 5,866,336 and 6,117,635).

The SNPs may also be detected using a mismatch detection technique, including but not limited to the RNase protection method using riboprobes (Winter et al, Proc. Natl. Acad Sci. USA 82:7575, 1985; Meyers et al, Science 230:1242, 1985) and proteins which recognize nucleotide mismatches, such as the E. coli mutS protein (Modrich, P. Ann. Rev. Genet. 25:229-253, 1991). Alternatively, SNPs can be identified by single strand conformation polymorphism (SSCP) analysis (Orita et al., Genomics 5:874-879, 1989; Humphries et al., in Molecular Diagnosis of Genetic Diseases, R. Elles, ed., pp. 321-340, 1996) or denaturing gradient gel electrophoresis (DGGE) (Wartell et al., Nucl. Acids Res. 18:2699-2706, 1990; Sheffield et al., Proc. Nat. Acad. Sci. USA 86:232-236, 1989).

In some embodiments, a SNP described herein can be detected using a method based on mass spectrometry. Mass spectrometry takes advantage of the unique mass of each of the four nucleotides of DNA. SNPs can be unambiguously detected by mass spectrometry by measuring the differences in the mass of nucleic acids having SNP compared to the samples from the control subject lacking SNPs. MALDI-TOF (Matrix Assisted Laser Desorption Ionization-Time of Flight) mass spectrometry technology is preferred for extremely precise determinations of molecular mass, such as SNPs. Numerous approaches to SNP analysis have been developed based on mass spectrometry. Preferred mass spectrometry-based methods of SNP genotyping include primer extension assays, which can also be utilized in combination with other approaches, such as traditional gel-based formats and microarrays.

SNP genotyping is useful for numerous applications, including, but are not limited to, SNP-disease association analysis, disease predisposition screening, disease diagnosis, disease prognosis, disease progression monitoring, determining therapeutic strategies based on an individual's genotype, developing effective therapeutic agents (e.g., an anti-eNAMPT antibody) based on SNP genotypes associated with a disease or likelihood of responding to a drug, and stratifying a patient population for clinical trial for a treatment regimen.

### Methods of Treatment

Also provided are methods of treating a subject having or at risk of developing a condition associated with NAMPT expression. Some embodiments comprise identifying a subject having or at risk for developing a condition associated with NAMPT expression, and treating the subject so as to prevent or reduce the development or progression of the condition. In some embodiments, the NAMPT-associated condition is an inflammatory condition, such as acute respiratory distress syndrome (ARDS), radiation-induced lung injury (RILI), pulmonary hypertension, or pulmonary fibrosis. In some embodiments, the NAMPT-associated condition is prostate cancer.

In some embodiments, the subject is treated by administering a NAMPT inhibitor to the subject (e.g., to reduce levels of NAMPT and/or reduce NAMPT activity). In some embodiments, a NAMPT inhibitor is an anti-NAMPT antibody. An exemplary anti-NAMPT antibody comprises Ab1 and Ab2, or antigen binding portions thereof, described in Table 3 below.

TABLE 3
Exemplary anti-NAMPT antibody VH/VL and CDR sequences

| | | | Sequence |
|---|---|---|---|
| Description | | Sequence | identifier |
| AB 1 | AB 1 heavy chain | QVQLVQSGAEVTKPGASVKVSCKASGYT | SEQ ID NO: 2 |
| | variable region | FTSYWMQWVRQAPGQGLEWVGEIDPSN | |
| | (VH) | SYTNYNQKFRGRVTLTRDTSTTTVYMEL | |
| | (CDRs underlined) | SSLRSEDTAVYYCARGGYWGQGTTVTVS | |
| | | S | |
| | AB 1 light chain | DIVMTQSPLSLPVTPGEPASISCRSSKSLL | SEQ ID NO: 3 |
| | variable region | HSQGITYLYWYLQKPGQSPQLLIYQLSNR | |
| | (VL) | ASGVPDRFSGSGSGTDFTLKISRVEAEDV | |
| | (CDRs underlined) | GVYYCVQNLELPYTFGGGTKLEIK | |
| | AB 1 CDR-H1 | GYTFTSYWMQ | SEQ ID NO: 4 |
| | AB 1 CDR-H2 | EIDPSNSYTNYNQKFRG | SEQ ID NO: 5 |
| | AB 1 CDR-H3 | ARGGY | SEQ ID NO: 6 |
| | AB 1 CDR-L1 | RSSKSLLHSQGITYLY | SEQ ID NO: 7 |
| | AB 1 CDR-L2 | QLSNRAS | SEQ ID NO: 8 |
| | AB 1 CDR-L3 | VQNLELPYT | SEQ ID NO: 9 |
| AB 2 | AB 2 heavy chain | EVQLVQSGAEVKKPGESLRISCKASGYTF | SEQ ID NO: 10 |
| | variable region | TSYWMHWVRQMPGKGLEWMGEIDPSDS | |
| | (VH) | YTNYNQKFKGHVTISADKSISTAYLQWSS | |
| | (CDRs underlined) | LKASDTAMYYCAKSNYVVPWYFDVWG | |
| | | QGTLVTVSS | |
| | AB 2 light chain | EIVLTQSPGTLSLSPGERATLSCRSSKSLL | SEQ ID NO: 11 |
| | variable region | HSNGITYLYWYQQKPGQAPRLLIYQMSN | |
| | (VL) | LASGIPDRFSGSGSGTDFTLTISRLEPEDFA | |
| | (CDRs underlined) | VYYCAQNLELPWTFGGGTKLEIK | |
| | AB 2 CDR-H1 | GYTFTSYWMH | SEQ ID NO: 12 |
| | AB 2 CDR-H2 | EIDPSDSYTNYNQKFKG | SEQ ID NO: 13 |
| | AB 2 CDR-H3 | AKSNYVVPWYFDV | SEQ ID NO: 14 |
| | AB 2 CDR-L1 | RSSKSLLHSNGITYLY | SEQ ID NO: 15 |
| | AB 2 CDR-L2 | QMSNLAS | SEQ ID NO: 16 |
| | AB 2 CDR-L3 | AQNLELPWT | SEQ ID NO: 17 |

In some embodiments, ARDS treatment comprises administering one or more of lung-protective ventilation; respiratory support (e.g., oxygen supplementation; positive-pressure ventilation); fluids; nutritional supplements; antimicrobials; steroids (e.g., glucocorticoids such as methylprednisolone); pulmonary vasodilators (e.g., nitric oxide; prostaglandin); β₂-adrenergic agonists; prostaglandin E₁; activated protein C; antioxidants; omega-3 fatty acids; ketoconazole; lisofylline; recombinant human factor VIIa; IFNβ1α; granulocyte-macrophage colony-stimulating factor; and statins. In some embodiments, one or more of the above therapies is administering in combination with one or more eNAMPT inhibitors (e.g., antibodies).

Treatment of RILI includes administration of one or more of radioprotectors (e.g., amifostine; engineered nanoparticles such as Manganese Superoxide Dismutase-Plasmid Liposomes; genistein; berberine; and/or pentoxifylline); radiomitigators (e.g., methyl prednisone; ACE (angiotensin-converting enzyme) inhibitors; angiotensin-2 antagonists; curcumin; and/or growth factors such as Keratinocyte Growth Factor); and cell-based therapies (e.g., bone marrow derived mesenchymal stem cells; and/or induced pluripotent stem cells). In some embodiments, one or more of the above therapies is administering in combination with one or more eNAMPT inhibitors (e.g., antibodies).

Treatment of pulmonary hypertension includes administration of one or more of vasodilators; anticoagulants (e.g., warfarin); diuretics; oxygen; digoxin; endothelial receptor antagonists (e.g., bosentan; ambrisentan; and/or macitentan); phosphodiesterase 5 inhibitors (e.g., sildenafil; and/or tadalafil); prostaglandins (e.g., epoprostenol; iloprost; and/or treprostinil); soluble guanylate cyclase stimulators (e.g., riociguat); and calcium channel blockers (e.g., nifedipine; diltiazem; nicardipine; and/or amlodipine). In some embodiments, one or more of the above therapies is administering in combination with one or more eNAMPT inhibitors (e.g., antibodies).

Treatment of pulmonary fibrosis includes administration of one or more of immunosuppressants (e.g., prednisolone; and/or azathioprine); antioxidants (e.g., N-acetylcysteine); antifibrotics (e.g., pirfenidone; and/or nintedanib); anti-acid medications; oxygen; connective tissue growth factor (CTFG) inhibitors; αvβ6 integrin inhibitors; autotaxin inhibitors; IL-13 inhibitors; and galectin-3 inhibitors. In some embodiments, one or more of the above therapies is administering in combination with one or more eNAMPT inhibitors (e.g., antibodies).

Treatments for COVID-19 include administering one or more of ACE inhibitors; angiotensin receptor blockers; remdesivir; oxygen; PIKfyve kinase inhibitors (e.g., apilimod); and cysteine protease inhibitors (e.g., MDL-28170; Z LVG CHN2; VBY-825; and/or ONO 5334). In some embodiments, one or more of the above therapies is administering in combination with one or more eNAMPT inhibitors (e.g., antibodies).

Treatment for prostate cancer includes surgery (e.g. to remove the prostate or testicles, or cryosurgery to kill cancer cells) and/or administering one or more of radiation therapy (e.g., external beam radiation; and/or brachytherapy); hormone therapy such as luteinizing hormone-releasing hormone (LH-RH) agonists (e.g., leuprolide; goserelin; triptorelin; and/or histrelin) or other medications to stop the body from producing testosterone (e.g., ketoconazole; and/or abiraterone); anti-androgens (e.g., bicalutamide; nilutamide; flutamide; and/or enzalutamide); chemotherapy; and biological therapy (e.g., sipuleucel-T). In some embodiments, one or more of the above therapies is administering in combination with one or more eNAMPT inhibitors (e.g., antibodies).

The below Examples further describe and demonstrate the compositions and methods of the present disclosure. The Examples are not intended to limit the disclosure in any way. Other aspects will be apparent to those skilled in the art. For example, in each instance herein any of the terms “comprising”, “consisting essentially of” and “consisting of” may be replaced with either of the other two terms; moreover, any of the terms may be used in reference to features disclosed herein.

### EXAMPLES

### Example 1

### Identification of SNPs Associated with Prostate Cancer

NAMPT promoter SNPs have been identified as indicators that may be used to identify patients having an increased risk and/or severity for prostate cancer.

12 NAMPT SNPs were reviewed and refined for assessing risk for prostate cancer progression, with several significantly over-represented in African descent individuals. NAMPT SNPs that contribute to ARDS susceptibility and mortality, were also identified. The SNPs are, rs7789066 (position: chr7:106287306 (GRCh38.p12)), rs116647506 (position: chr7:106287180 (GRCh38.p12)), rs61330082 (position: chr7:106286419 (GRCh38.p12)), rs114382471 (position: chr7:106286288 (GRCh38.p12)), rs9770242 (position: chr7:106285885 (GRCh38.p12)), rs59744560 (position: chr7:106285832 (GRCh38.p12)), rs190893183 (position: chr7:106285663 (GRCh38.p12)), and rs1319501 (position: chr7:106285307 (GRCh38.p12)).

These NAMPT SNPs contribute to ARDS susceptibility subsequently replicating and altering NAMPT promoter activity in response to mechanical stress and to hypoxia with key involvement by hypoxia-induced transcription factor HIF2α and significantly influenced by NAMPT promoter SNPs -948T, -1001G, and -2422G, but not by -1535G, which are protective SNPs in ARDS. Basal and radiation-induced NAMPT promoter activities in normal prostate cells (RWPE-1) and in PCa cells (PC3, DU-145) were evaluated. Basal NAMPT promoter activity is significantly greater in prostate cancer cells than normal prostate cells and further increased by radiation (8 Gy, 4 hrs), indicating a potential mechanism by which NAMPT expression may be stimulated in response to reactive oxygen species to promote prostate cancer progression.

### Example 2

### NAMPT Genotyping and Plasma eNAMPT Assays to Identify Risk for Aggressive Prostate Cancer

This Example illustrates NAMPT promoter SNPs and/or increase plasma levels of eNAMPT as biomarkers for aggressive prostate cancer. To demonstrate that elevated eNAMPT levels and/or the presence of NAMPT SNPs are associated with increased mortality and disease progression in prostate cancer, NAMPT genotyping assay panel, and plasma-based eNAMPT ELISA values in biobanked specimens from subjects previously enrolled in prostate cancer clinical trials, containing phenotypic information, including prostate biopsy results, PSA levels, bone scans, and MRI prostate imaging, will be evaluated. The subjects were enrolled in Phase II/III PCa studies and include 166 individuals with initially negative biopsies for prostate cancer and ˜20 plasma samples obtained over a 3-5-year period. Over half of this cohort (55%) eventually developing biopsy-proven prostate cancer, and therefore, provide an invaluable set of specimens for determining if NAMPT SNPs and eNAMPT levels predict risk of and progression of PCa. A second set of paired DNA and multiple plasma samples obtained from African-American subjects will also be evaluated to determine disease predicting factors that result in prostate cancer progression and lethality.

### Example 3

### NAMPT Expression in Human Invasive PCa

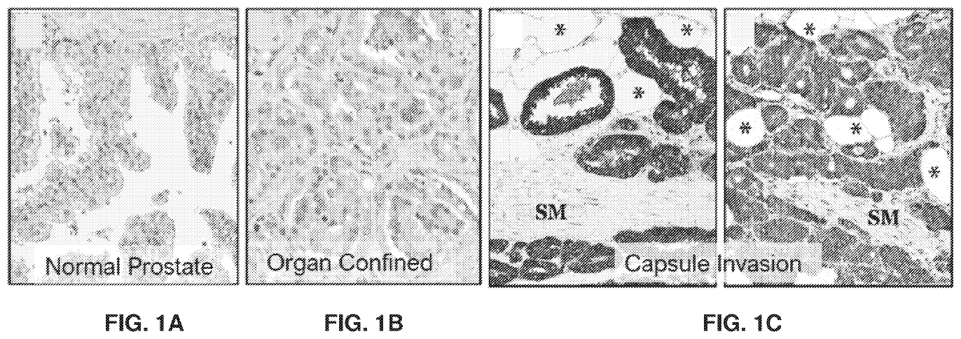

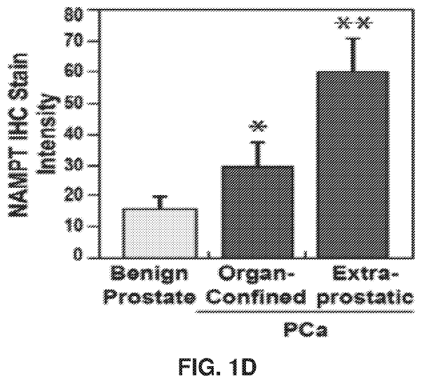

To assess the role of NAMPT in PCa invasiveness and progression, NAMPT expression was studied in PCa tissue. Expression of NAMPT was assessed by immunohistochemical (IHC) staining in normal prostate tissue, in prostatic adenocarcinoma confined to the prostate and without capsular invasion (i.e., organ-confined PCa), and in prostatic adenocarcinomas with capsular invasion into extra-prostatic adipose tissues (i.e., invasive PCa). Representative micrographs are provided in FIGS. 1A-1C. FIG. 1D summarizes NAMPT staining as assessed from the micrographs of FIGS. 1A-1C.

IHC analysis of the normal and PCa tissues showed virtual absence of NAMPT expression in normal prostate tissue (FIGS. 1A and 1D), and considerable expression of NAMPT in prostatic adenocarcinoma confined to the prostate and without capsular invasion (FIGS. 1B and 1D; p<0.05). In contrast, prostatic adenocarcinomas with capsular invasion into extra-prostatic adipose tissues showed significantly robust NAMPT staining (FIGS. 1C and 1D; p<0.005).

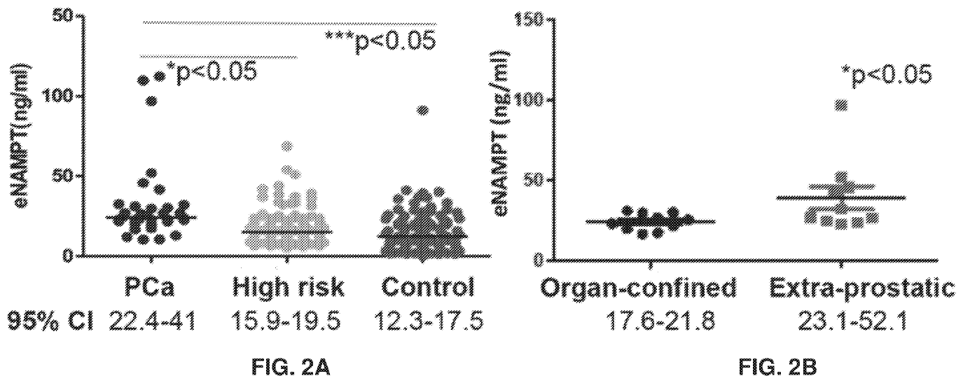

To further assess the role of NAMPT in PCa invasiveness and progression, extracellular NAMPT expression was evaluated by ELISA in plasma samples obtained from healthy controls, PCa patients and high risk subjects who exhibit elevated PSA levels but who are negative on prostate biopsies. Results from the analyses are provided in FIG. 2A. As shown in FIG. 2A, NAMPT plasma level was higher in PCa patients compared to high risk subjects (p<0.05). Moreover, NAMPT plasma level in PCa patients and in high risk patients was found to be higher when compared to that from healthy controls (p<0.05). Additionally, NAMPT plasma levels of patients with organ-confined PCa (or non-invasive PCa) and patients with extra-prostatic PCa (or invasive PCa) were compared. The comparative analysis is provided in FIG. 2B, which shows that NAMPT plasma level was significantly higher in patients with extra-prostatic or invasive PCa compared to patients with organ-confined or non-invasive PCa (p<0.05).

Thus the results outlined in FIGS. 1A-1D, 2A and 2B show increased expression of NAMPT in invasive PCa, underscoring a critical role of NAMPT in PCa invasiveness.

### Example 4

### Effect of Humanized Anti-NAMPT Antibody on PCa Cell Invasion

The results outlined in the foregoing example indicate a role of NAMPT in PCa invasiveness and underscore NAMPT as a potential therapeutic target in invasive PCa. To further validate the role of NAMPT as a therapeutic target in invasive PCa, the effect of a humanized anti-NAMPT monoclonal antibody (mAb) on PCa cell invasion was evaluated. To this end, peritoneal invasion of human PCa cells was evaluated in severe combined immunodeficient (SCID) mice.

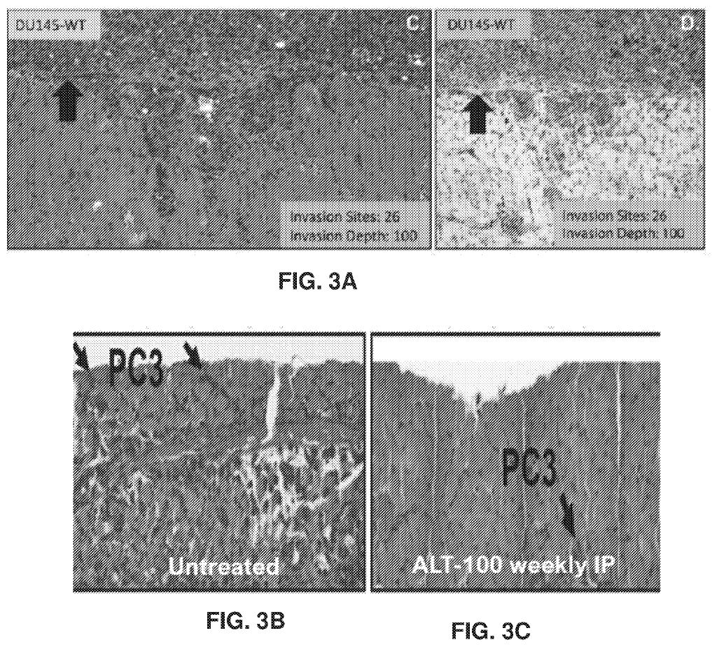

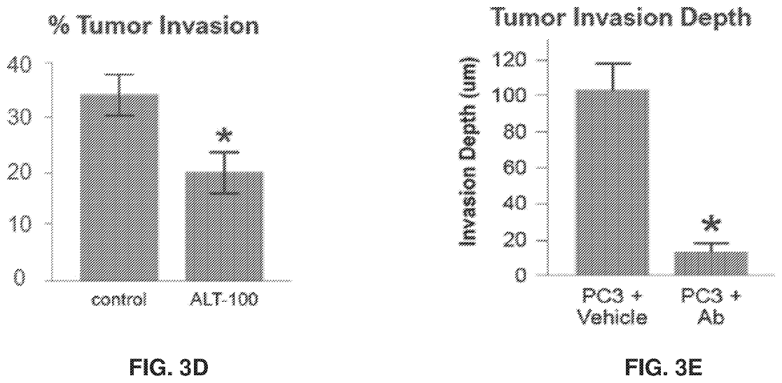

First, metastatic human PCa cells, PC3, were injected intraperitoneally (I.P.) into SCID mice. In specific experiments, mice were also injected two times a week with 2 μg of humanized anti-NAMPT mAb or vehicle alone. Peritoneal invasion of the PC3 cells was evaluated 6 weeks after the PC3 cell injection. Representative micrographs are provided in FIGS. 3A-3C, and graphical summary of the results are provided in FIGS. 3D and 3E.

As depicted in FIGS. 3A-3C, injection of human PCa cell line caused substantial peritoneal muscle invasion (FIGS. 3A and 3B) associated with prominent NAMPT staining within the invading cancer cells, while PC3-challenged SCID mice receiving humanized anti-NAMPT mAb exhibited marked reductions in PC3 invasion of the smooth muscle peritoneum (FIG. 3C). As summarized in FIGS. 3D and 3E, PC3-challenged SCID mice receiving humanized anti-NAMPT mAb showed significantly reduced tumor invasion percentage (FIG. 3D, p<0.05) and tumor invasion depth (FIG. 3E, p<0.05).

Thus, observations from this study strongly implicate a role for NAMPT in PCa cell invasiveness and a critical potential for humanized anti-NAMPT antibody to temporize this invasive behavior.

### Example 5

### Plasma NAMPT Levels as a Diagnostic/Prognostic Biomarker in Human ARDS

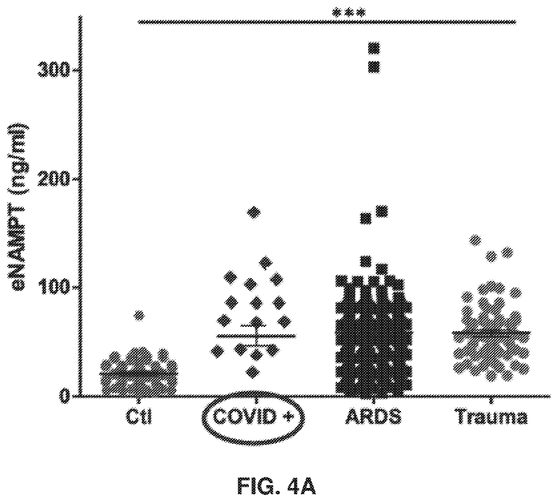

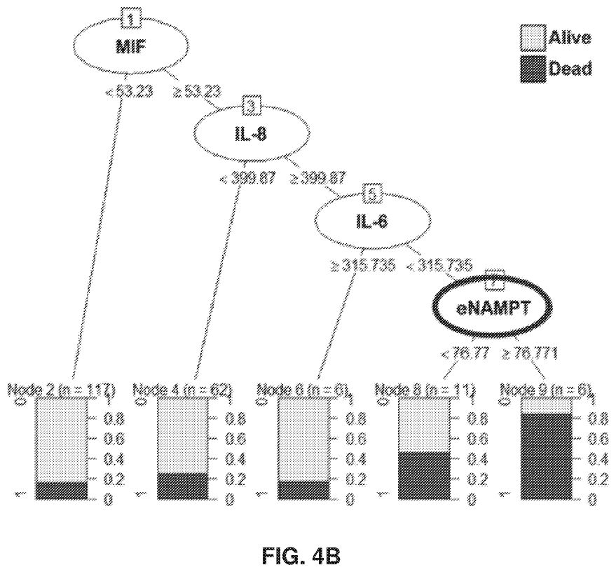

Plasma samples were obtained from patients with COVID-19 infection, ARDS or trauma or from healthy controls (“Ctl”), and NAMPT level in the plasma samples was assessed by ELISA. The results are shown in FIG. 4A, in which plasma NAMPT level was significantly higher (p<0.001) in patients with acute inflammatory conditions, such as COVID-19 infection, ARDS or trauma, compared to that from healthy controls. Next, the plasma levels of NAMPT and other inflammatory cytokines, such as IL-6, IL-8, and macrophage migration inhibitory factor (MIF) in alive and dead ARDS patients was evaluated to assess the correlation between plasma levels of these inflammatory cytokines and ARDS mortality. The result is shown in FIG. 4B, which depicts a higher plasma level of NAMPT and other inflammatory cytokines, such as IL-6, IL-8, and MIF in dead ARDS patients, showing that higher plasma levels of these cytokines is associated with ARDS mortality.

Accordingly, the results show a dysregulation of NAMPT expression in ARDS and other acute inflammatory conditions and indicate the potentials of NAMPT as a diagnostic/prognostic biomarker in ARDS.

### Example 6

### Plasma NAMPT Levels as a Diagnostic/Prognostic Biomarker in Pancreatitis

Example 5 establishes NAMPT as a diagnostic/prognostic biomarker in ARDS and other inflammatory conditions. Considering the fact that pancreatitis, a condition characterized by parenchymal inflammation of the pancreas, is often associated with ARDS, we next assessed the potentials of NAMPT as a diagnostic/prognostic biomarker in pancreatitis.

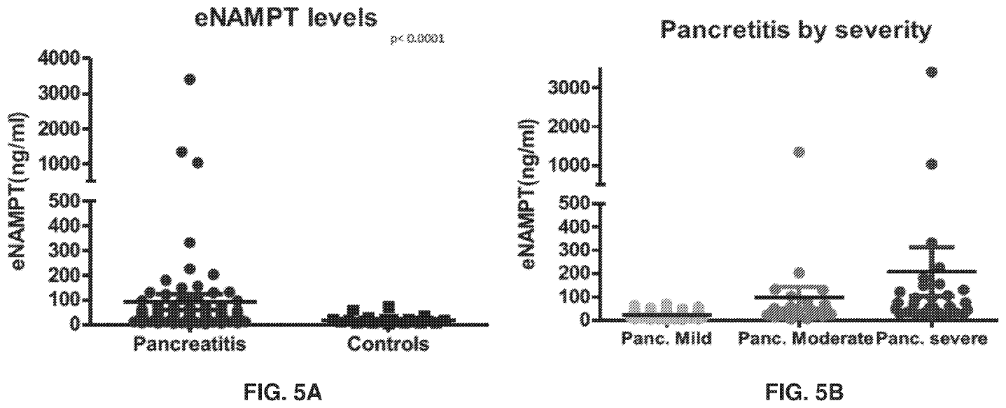

To this end, first, NAMPT plasma level was evaluated by ELISA in samples obtained from pancreatitis patients and healthy controls. The results are shown in FIG. 5A, which indicates that compared to healthy controls, plasma NAMPT level was significantly higher (p<0.0001) in patients with pancreatitis, indicating dysregulation of NAMPT expression in pancreatitis. Next, NAMPT plasma level was evaluated by ELISA in samples obtained from patients with mild, moderate or severe pancreatitis. As shown in FIG. 5B, plasma NAMPT level was markedly higher in patients with moderate pancreatitis compared to those with mild pancreatitis, while patients with severe pancreatitis showed even higher plasma NAMPT levels. Thus, FIG. 5B shows an increase in plasma NAMPT with increase in pancreatitis severity.

Accordingly, the results outlined in FIGS. 5A and 5B demonstrate a role of NAMPT in pathogenesis and progression of pancreatitis and underscore the potentials of NAMPT as a diagnostic/prognostic biomarker of pancreatitis and pancreatitis associated ARDS.

### Example 7

### Plasma NAMPT Levels as a Diagnostic/Prognostic Biomarker in Sepsis

Example 5 establishes NAMPT as a diagnostic/prognostic biomarker in ARDS. Considering the fact that severe sepsis is the most common etiology of ARDS, we next assessed the potentials of NAMPT as a diagnostic/prognostic biomarker in sepsis.

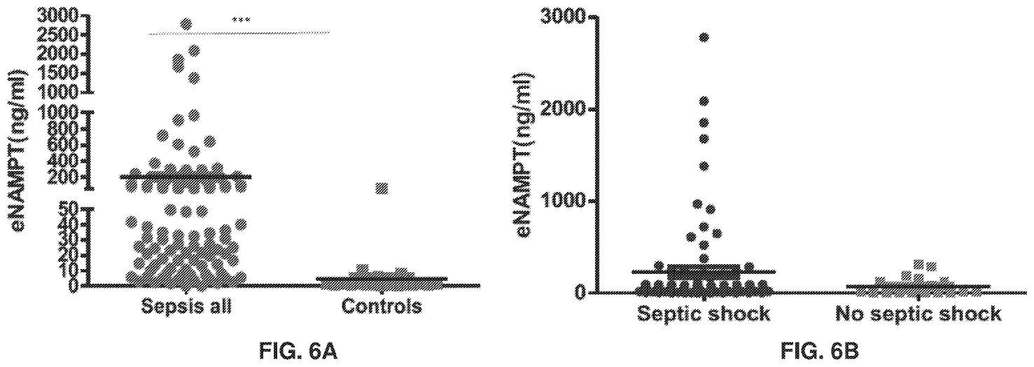

NAMPT plasma level was evaluated by ELISA in samples obtained from healthy controls or patients with sepsis. As shown in FIG. 6A, compared to healthy controls, plasma NAMPT level was significantly higher (p<0.001) in sepsis patients, indicating dysregulation of NAMPT expression in sepsis. Next, NAMPT plasma level was evaluated by ELISA in samples obtained from sepsis patients with or without septic shock. As shown in FIG. 6B, plasma NAMPT level was markedly higher in sepsis patients with septic shock compared to sepsis patients without septic shock. These results show increase in plasma NAMPT level with increase in sepsis severity. Accordingly, the data demonstrate a role of NAMPT in pathogenesis and progression of sepsis and underscore the potentials of NAMPT as a diagnostic/prognostic biomarker of sepsis and sepsis-induced ARDS.

### Example 8

### NAMPT Genetic Variants Predict ARDS Severity

Single nucleotide polymorphisms (SNPs) in genes regulate cytokines such as NAMPT. The presence of certain SNPs is an indication of the presence of extracellular NAMPT. As the foregoing examples establish plasma NAMPT as a diagnostic/prognostic biomarker in ARDS, we next evaluated if detection and measurement of certain (SNPs) in genes can be used to detect NAMPT and correlate to a risk for ARDS. To this end, DNA samples from ARDS patients or healthy controls were evaluated for certain SNPs associated with NAMPT promoter.

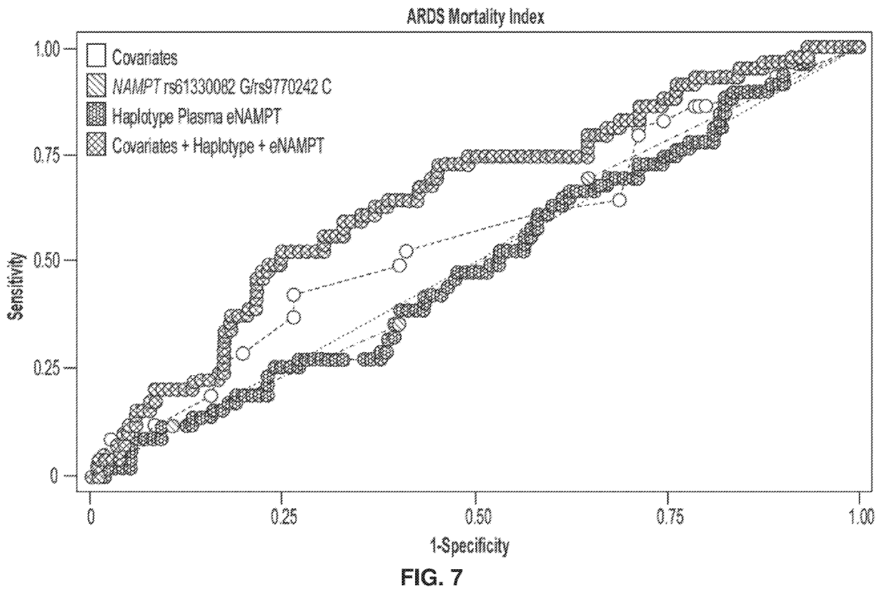

As shown in FIG. 7, NAMPT SNPs rs61330082 and rs9770242 were found to be associated with higher plasma NAMPT level and higher ARDS mortality. Moreover, ARDS mortality index was found to integrate plasma NAMPT level, NAMPT SNPs and clinical covariates genotypes. Thus, the results described in FIG. 7 demonstrate that NAMPT genotypes could effectively predict higher plasma NAMPT levels and higher ARDS mortality.

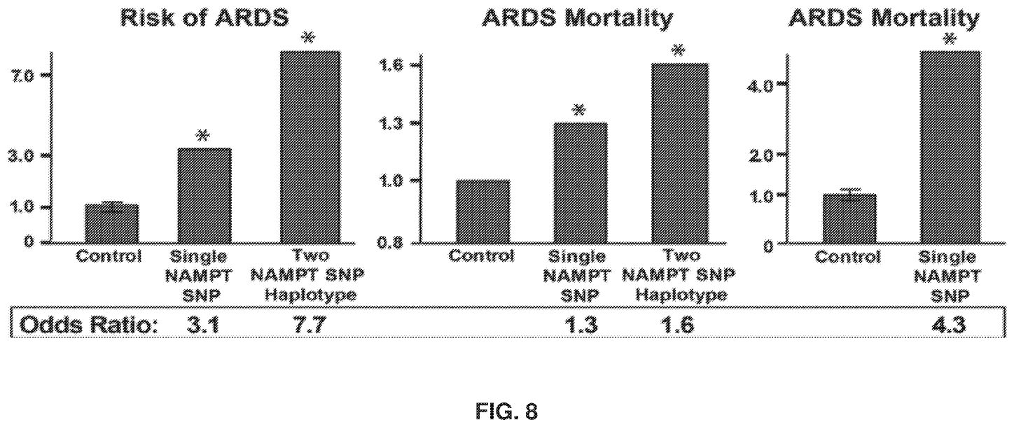

Furthermore, NAMPT sequencing identified 5 SNPs that confer increased risk of developing ARDS, including SNPs over-represented in African descent subjects. To establish the correlation of NAMPT SNPs to ARDS risk and ARDS mortality, ARDS patients with single NAMPT SNP and two NAMPT SNPs were compared to control ARDS patients (“Control”) with no NAMPT SNPs (i.e., ARDS patients with wild-type NAMPT allele). The results are described in FIG. 8. As shown in FIG. 8, NAMPT SNPs showed significant correlation to risk of ARDS and ARDS mortality over control (p<0.05). Also, a haplotype with two NAMPT SNPs showed higher correlation to risk of ARDS (FIG. 8, left panel) and ARDS mortality (FIG. 8, middle panel) compared to a haplotype with single NAMPT SNP. Thus, the results described in FIG. 8 demonstrate that “high risk” NAMPT genotypes (SNPs) can effectively predict risk of ARDS and ARDS severity and mortality.

Accordingly, NAMPT genetic variants (SNPs) can be effective in predicting NAMPT plasma levels, and eventually risk of ARDS and ARDS mortality.

### Example 9

### Assessing the Effect of Radiation on NAMPT Expression Using an In Vivo Model of Radiation Pneumonitis

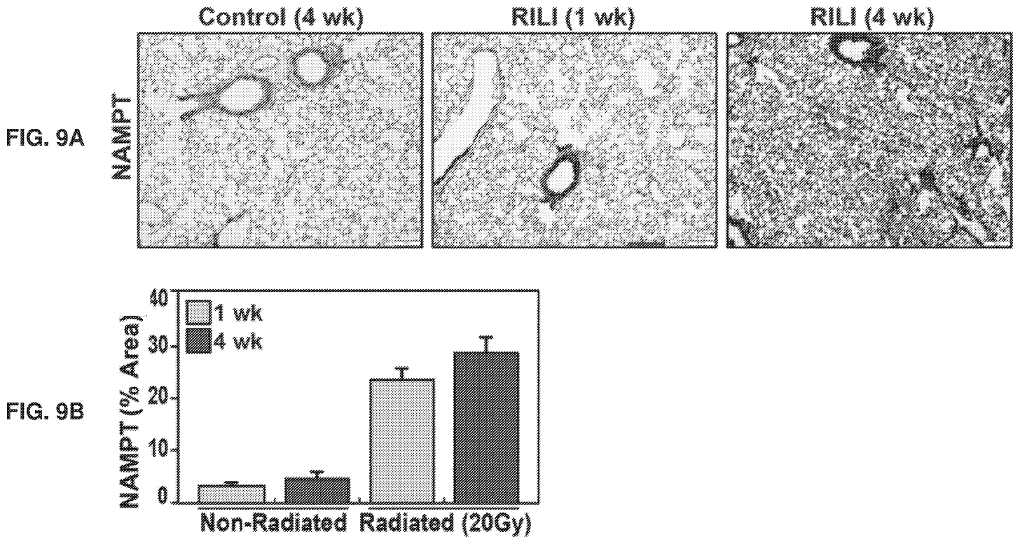

In order to assess the role of NAMPT in RILI, the effect of radiation on NAMPT expression was studied. To this end, WT C57/B6 mice were exposed to 20 Gy whole thorax lung irradiation (WTLI) and evaluated at specified time points over a 4-week period. The results are described in FIGS. 9A-B.

As shown in FIG. 9A, WTLI-exposed WT mice exhibited increased NAMPT expression, especially in alveolar macrophages and epithelial cells, and an increase in inflammation, vascular leakage and inflammatory lung injury 1 week (FIG. 9A, middle panel) and 4 weeks (FIG. 9A, right panel) after 20 Gy WTLI, compared to control mice (non-irradiated mice; shown in FIG. 9A, left panel). FIG. 9B summarizes NAMPT staining in lung tissues of WTLI-exposed mice 1 week and 4 weeks after IR exposure, or in lung tissues of control mice (non-irradiated mice).

Thus, the results show radiation-induced increase in NAMPT expression, which indicates a role for NAMPT in RILI pathogenesis and the potentials of using NAMPT as a biomarker of RILI.

### Example 10

### Effect of Radiation on NAMPT Expression in Human Tissues and Blood

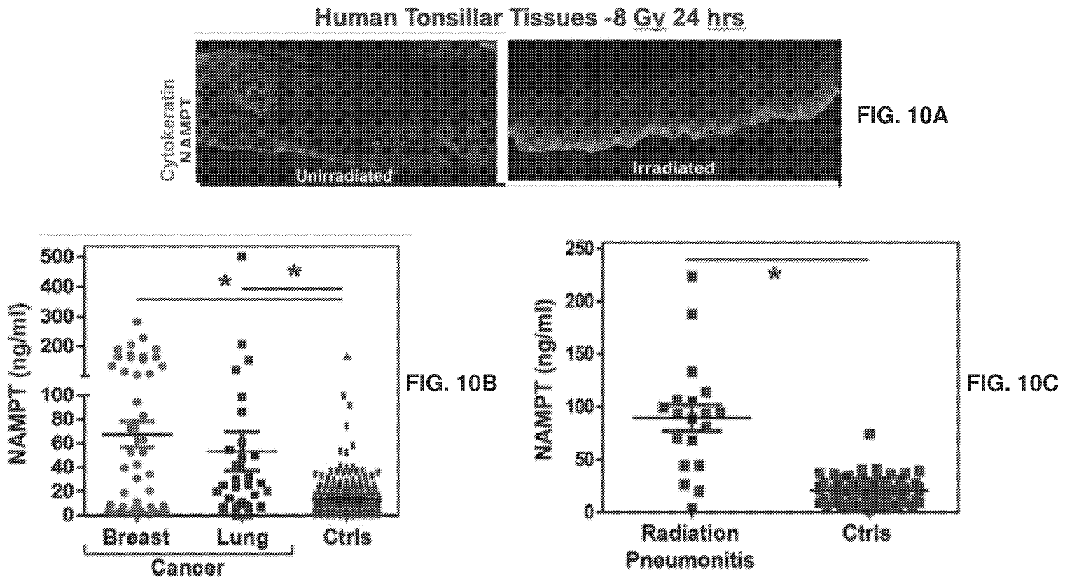

To further explore the role of NAMPT in RILI, the effects of radiation on expression of NAMPT in human tissues and blood was explored. The results are described in FIGS. 10A-C.

To assess the effect of radiation on NAMPT expression, human tonsillar epithelial tissue was exposed to 8 Gy ionizing radiation (IR) for 24 hours. As shown in FIG. 10A, NAMPT expression in human tonsillar tissues was rapidly and markedly upregulated after 8 Gy IR exposure. The effect of radiation on NAMPT expression was further assessed by studying NAMPT expression in cancer patients undergoing radiotherapy. As shown in FIG. 10B, subjects undergoing radiotherapy for breast cancer or lung cancer exhibited significantly increased plasma level of NAMPT compared to control subjects (p<0.05). The effect of radiation on NAMPT expression was also assessed by studying NAMPT expression in patients with radiation pneumonitis. As described in FIG. 10C, patients with radiation pneumonitis exhibited NAMPT plasma level that was 4-5 fold higher than control subjects (p<0.05).

Thus, the results indicate a dysregulation of NAMPT expression and secretion in human RILI.

### Example 11

### Exploring the Role of NAMPT in RILI

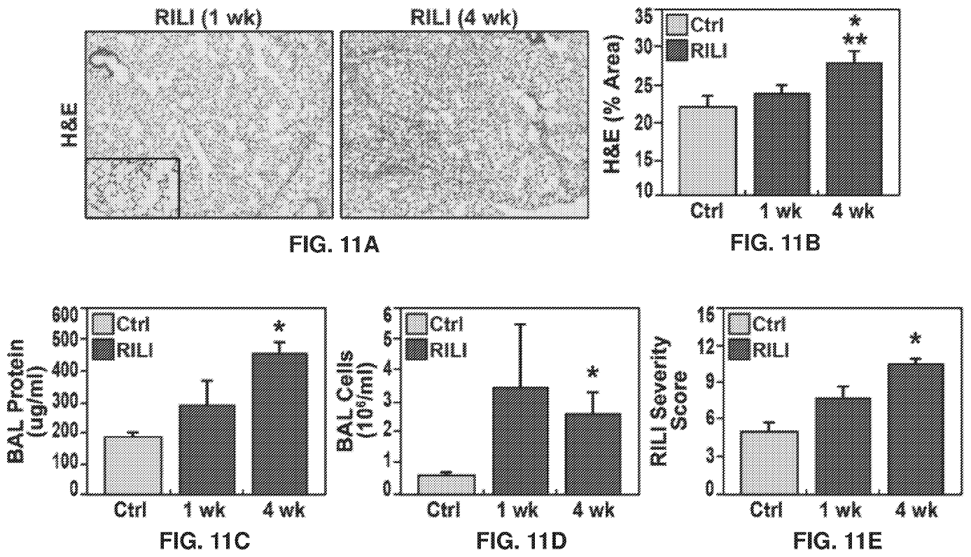

The role of NAMPT in RILI was further assessed using in vivo experiments in C57/B6 mice. A first group of mice consisted of wild type (WT) mice receiving 20 Gy thoracic radiation. Non-irradiated mice served as negative control (“Control” or “Ctrl”). Lung tissues were harvested from the mice at specific times over a 4-week period. Amount of bronchoalveolar lavage (BAL) protein was measured and count of BAL-expressing cells was obtained. Lung tissues were also subjected to hematoxylin and eosin (H&E) staining to assess lung inflammation. Moreover, RILI severity score was assessed based on BAL indices and H&E staining. Results from the corresponding analyses are provided in FIGS. 11A-E.

As shown in FIG. 11A, development of RILI in mice was confirmed by H&E staining of lung tissues that displayed acute diffuse alveolar damage 1 week (FIG. 11A, left panel) and 4 weeks (FIG. 11A, right panel) after radiation exposure, compared to lung tissues from non-irradiated controls (inset of FIG. 11A, left panel). FIG. 11B summarizes H&E staining in lung tissues of irradiated mice 1 week and 4 weeks after IR exposure compared to that in lung tissues of non-irradiated control mice. As shown in FIG. 11B, a significant increase in H&E stained area was seen in lung tissues of irradiated mice 4 weeks after IR exposure (p<0.001), suggesting effective development of RILI. FIG. 11C shows BAL protein levels in lung tissues of irradiated mice 1 week and 4 weeks after IR exposure compared to that in lung tissues of non-irradiated control mice. As shown in FIG. 11C, compared to control mice, mice that were exposed to radiation displayed increased BAL protein levels beginning at week 1 post radiation exposure, with significant increase in BAL protein levels seen 4 weeks after irradiation (p<0.05). Similarly, as shown in FIG. 11D, count of BAL-expressing cells (BAL cells) increased in mice that were exposed to irradiation, with a significant increase in BAL cell count observed 4 weeks after radiation exposure (p<0.05). Furthermore, as shown in FIG. 11E, compared to control mice, mice that were exposed to radiation displayed increased RILI severity score beginning at week 1 post radiation exposure, with significant increase in RILI severity score observed 4 weeks after irradiation (p<0.05).

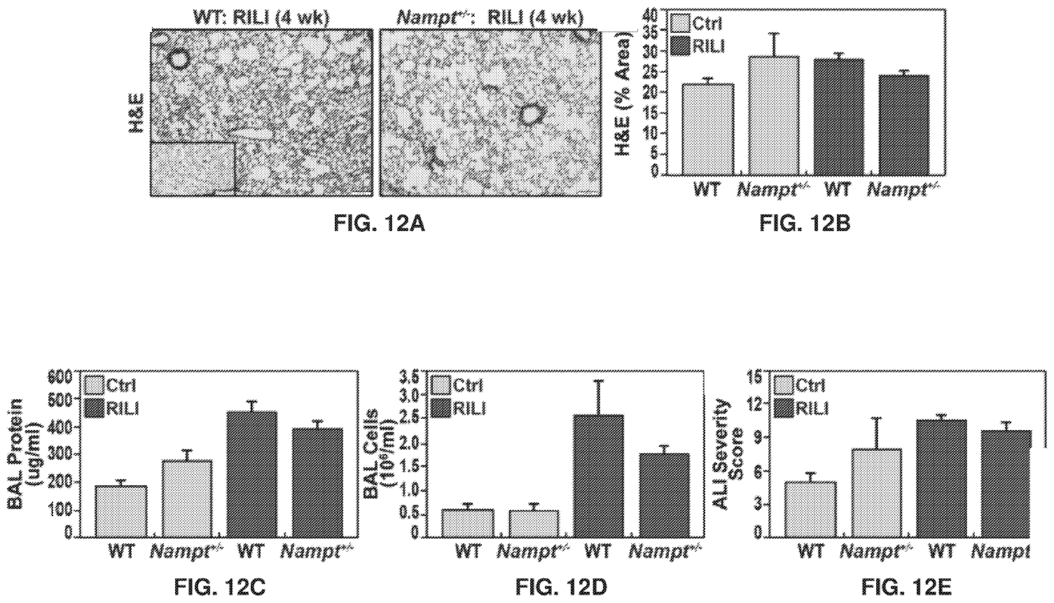

A second group consisted of NAMPT heterozygous (Nampt^{+/-}; “Nampt het”) mice that received 20 Gy thoracic radiation and were observed for 4 weeks. Non-irradiated WT and NAMPT heterozygous mice, and irradiated WT mice were used as controls. Amount of BAL protein was measured and count of BAL-expressing cells was obtained. Lung tissues were also subjected to H&E staining to assess lung inflammation. Moreover, acute lung injury (ALI) severity score was assessed based on BAL indices and H&E staining. Results from the corresponding analyses are provided in FIGS. 12A-E.

As shown in FIG. 12A, H&E staining of lung tissues from WT irradiated mice displayed diffuse alveolar damage 4 weeks after radiation exposure (FIG. 12A, left panel), compared to lung tissues from non-irradiated WT mice (inset of FIG. 12A, left panel). In contrast, lung tissues from Nampt^{+/-} mice (FIG. 12A, right panel) demonstrated reduced H&E staining, indicating less alveolar damage in Nampt^{+/-} mice following radiation exposure. FIG. 12B summarizes H&E staining in lung tissues of irradiated or non-irradiated WT and Nampt^{+/-} mice. As shown in FIG. 12B, reduced H&E stained area was observed in lung tissues from irradiated Nampt mice compared to that from irradiated WT mice, thus indicating a role of NAMPT in pathogenesis of RILI. FIG. 12C shows BAL protein levels in lung tissues of irradiated or non-irradiated WT and Nampt^{+/-} mice. As shown in FIG. 12C, compared to non-irradiated control mice, mice that were exposed to radiation displayed increased BAL protein levels. However, irradiated Nampt^{+/-} mice demonstrated reduced BAL protein level compared to the irradiated WT control. Similarly, count of BAL cells increased in mice that were exposed to irradiation, although irradiated Nampt^{+/-} mice demonstrated markedly reduced BAL cell count compared to the irradiated WT control. Furthermore, as described in FIG. 12E, compared to control mice, mice that were exposed to radiation displayed increased ALI severity score; however, irradiated Nampt^{+/-} mice demonstrated reduced ALI severity score compared to the irradiated WT control. Thus, the results show reduced manifestation of RILI in Nampt^{+/-} mice, indicating a role of NAMPT in development and progression of RILI.

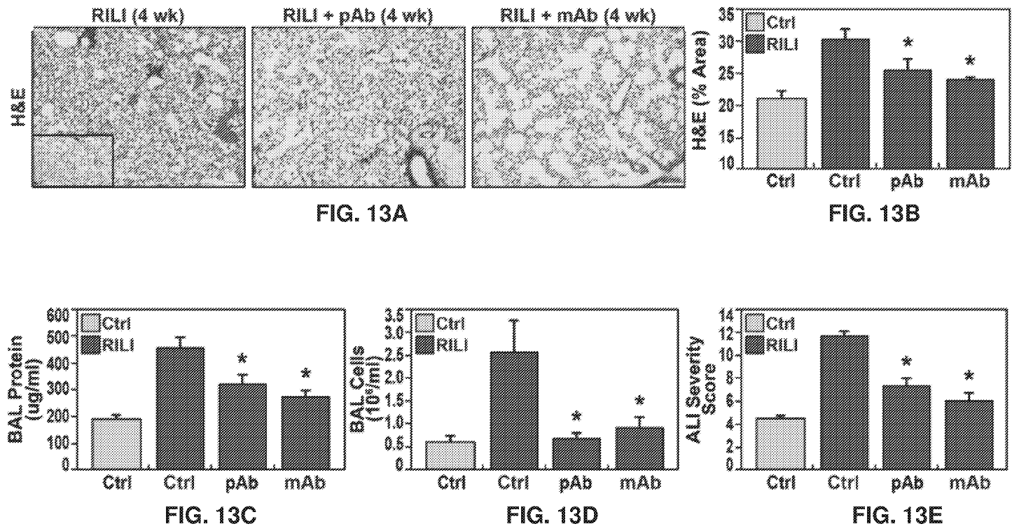

A third group consisted of radiated mice that received 20 Gy thoracic radiation and were injected intraperitoneally with a polyclonal NAMPT-neutralizing antibody (pAb) or a monoclonal anti-NAMPT antibody (mAb). Non-irradiated mice and irradiated mice injected with vehicle alone were used as controls (“Ctrl”). Amount of BAL protein was measured and count of BAL-expressing cells was obtained. Lung tissues were also subjected to H&E staining to assess lung inflammation. Moreover, acute lung injury (ALI) severity score was assessed based on BAL indices and H&E staining. Results from the corresponding analyses are provided in FIGS. 13A-E.

As described in FIG. 13A, H&E staining of lung tissues from irradiated control mice (injected with vehicle alone) displayed diffuse alveolar damage 4 weeks after radiation exposure (FIG. 13A, left panel), compared to lung tissues from non-irradiated control mice (inset of FIG. 13A, left panel). In contrast, lung tissues from mice that were injected with anti-NAMPT pAb (FIG. 13A, middle panel) or anti-NAMPT mAb (FIG. 13A, right panel) demonstrated reduced H&E staining, indicating less alveolar damage in anti-NAMPT Ab treated mice following radiation exposure. FIG. 13B summarizes H&E staining in lung tissues of non-irradiated control mice, irradiated control mice, and irradiated mice that were injected with anti-NAMPT pAb or mAb. As shown in FIG. 13B, H&E stained area was increased in lung tissues from irradiated control mice compared to that from non-irradiated control mice. However, compared to irradiated control mice, a significant reduction in H&E stained area was observed in lung tissues from mice that were injected with anti-NAMPT pAb or mAb (p<0.05), suggesting a role of NAMPT in development of RILI. FIG. 13C shows BAL protein levels in lung tissues of non-irradiated control mice, irradiated control mice, and irradiated mice that were injected with anti-NAMPT pAb or mAb. As shown in FIG. 13C, compared to non-irradiated control mice, mice that were exposed to radiation displayed increased BAL protein levels. However, irradiated mice that were injected with anti-NAMPT pAb or mAb demonstrated significantly reduced BAL protein level compared to the irradiated control mice (p<0.05), with more pronounced reduction observed in irradiated mice that were treated with anti-NAMPT mAb. Similarly, count of BAL cells increased in mice that were exposed to irradiation, although irradiated mice that were injected with anti-NAMPT pAb or mAb demonstrated markedly reduced BAL cell count compared to the irradiated control mice (p<0.05), with more pronounced reduction observed in irradiated mice that were treated with anti-NAMPT mAb. Furthermore, as shown in FIG. 13E, compared to control mice, mice that were exposed to radiation displayed increased ALI severity score; however, irradiated mice that were injected with anti-NAMPT pAb or mAb demonstrated significantly reduced ALI severity score compared to the irradiated control mice, with more pronounced reduction observed in irradiated mice that were treated with anti-NAMPT mAb. Thus, the results described in FIGS. 13A-E show attenuation of RILI following treatment with anti-NAMPT Abs, underscoring NAMPT as a potential therapeutic target in RILI.

Thus, the results demonstrate a dysregulation of NAMPT expression and secretion in RILI, and indicate that NAMPT is a novel biomarker and therapeutic target in RILI that contributes to the pathobiology of radiation-induced injury in lung tissues.

### Example 12

### Radiolabeled Anti-NAMPT Antibody Identifies Increased NAMPT Expression in Inflamed Lung Tissues

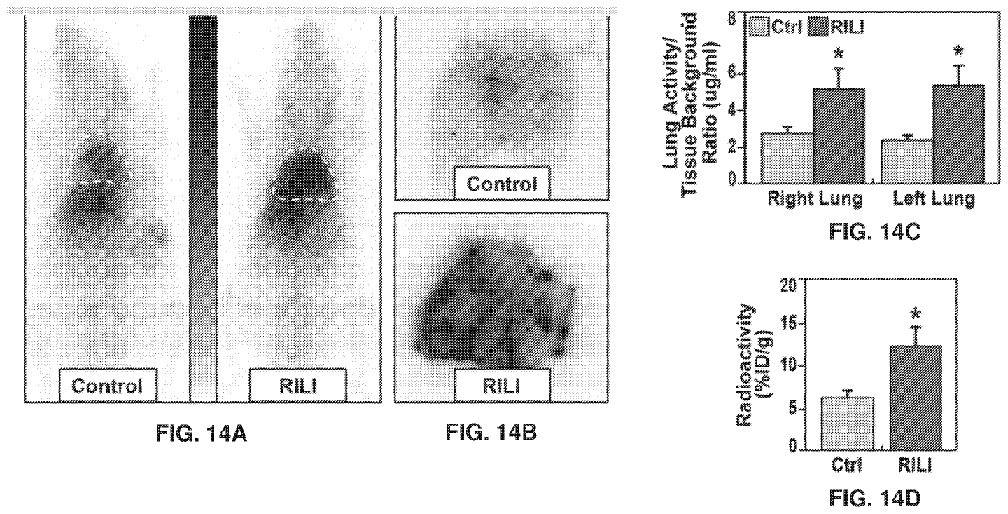

Radiolabeled anti-NAMPT antibodies were developed with the goal of non-invasively detecting NAMPT signaling pathway and NAMPT expression in different tissues in vivo. Imaging the mouse models with RILI using radiolabeled anti-NAMPT mAb would enable defining the optimal time for deploying anti-NAMPT mAb as a therapeutic intervention and to survey the major organs for inflammation and cellular apoptosis, employing other specific radiolabels, following total body irradiation (TBI) or partial body irradiation (PBI), such as in a nuclear incident. To test the detection of NAMPT expression by the radiolabeled anti-NAMPT antibody, ^{99m}Tc-labeled anti-NAMPT mAb probe was injected into control mice and mice that were exposed to 8 Gy PBI, and rapid autoradiograph imaging was performed. Results from the analysis are described in FIGS. 14A-D.

As shown in FIGS. 14A-B, higher radioactive uptake was observed in lungs of irradiated mice compared to non-irradiated control mice, indicating higher NAMPT expression induced by RILI. Furthermore, uptake of radiolabeled anti-NAMPT antibody was used as a measure of lung activity in irradiated mice or non-irradiated control mice. As shown in FIG. 14C, a significant increase in lung activity over tissue background was observed in both right and left lungs from irradiated mice compared to those from non-irradiated control mice (p<0.05). Moreover, level of radioactivity in irradiated mice or non-irradiated control mice was determined to assess uptake of the radiolabeled anti-NAMPT mAb. As shown in FIG. 14D, a significant increase in radioactivity was observed in irradiated mice compared to non-irradiated control mice (p<0.05), thus confirming increased uptake of the radiolabeled anti-NAMPT mAb in irradiated mice.

Thus, the radiolabeled anti-NAMPT antibody was effective in detecting increased NAMPT expression in inflamed lung tissues. This underscores the potentials of utilizing the radiolabeled anti-NAMPT antibody as a tool for detection of NAMPT, which could be pivotal in using NAMPT as a biomarker in RILI.

### Example 13

### Validating NAMPT as a Therapeutic Target in RILI Using an In Vivo Model of Radiation-Induced Lung Fibrosis

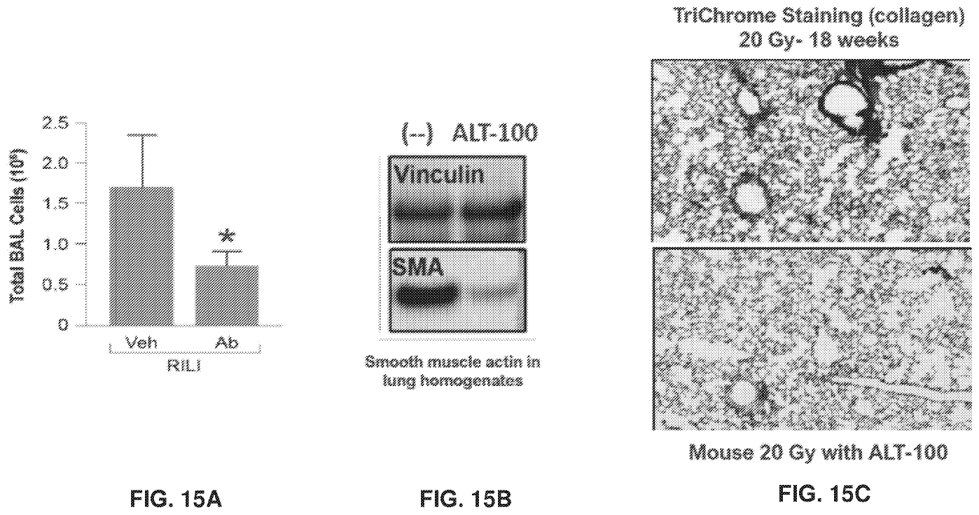

To further validate NAMPT as a therapeutic target in RILI, WT C57/B6 mice were exposed to 20 Gy WTLI. The irradiated mice were intraperitoneally injected with 10 μg of an anti-NAMPT mAb or vehicle control. The mice were evaluated for radiation-induced lung fibrosis (RILF) 18 weeks post radiation exposure by assessing BAL cell count, collagen deposition, and expression of lung tissue smooth muscle actin (SMA), which is a reflection of myofibroblast transition and fibrosis. The results are shown in FIGS. 15A-C.

As shown in FIGS. 15A-C, the anti-NAMPT mAb significantly reduced IR-induced RILI, which was reflected by decreased BAL cell count (FIG. 15A), decreased expression of lung tissue SMA (detected by western blot analyses, shown in FIG. 15B), and decreased collagen deposition (detected by Trichrome staining of lung tissues, shown in FIG. 15C) in Ab-treated mice compared to vehicle-treated control mice.

Thus, the results underscore the role of an anti-NAMPT Ab in attenuating RILF, further validating NAMPT as a therapeutic target in RILI.

### Example 14

### Evaluating the Efficacy of an Anti-NAMPT mAb in Pre-Clinical Models of Lung Injury

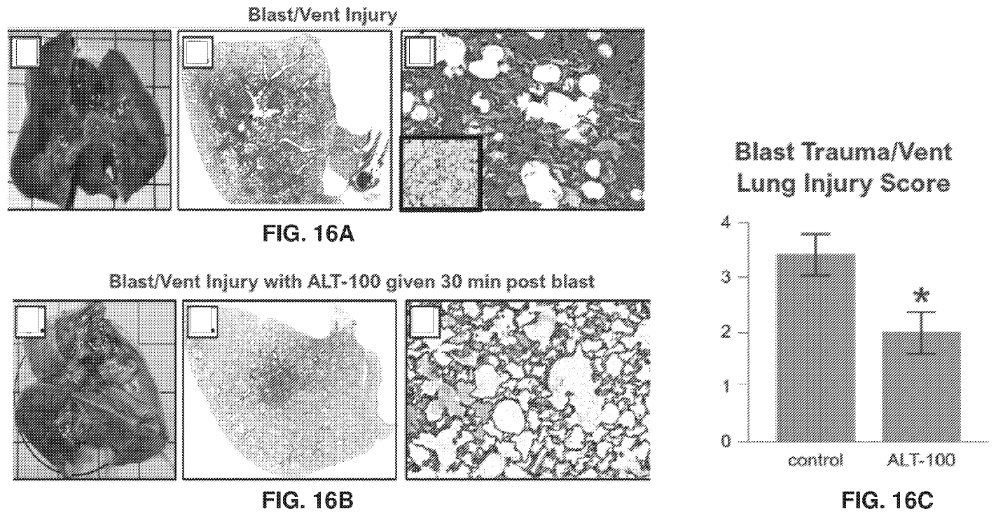

The efficacy of an anti-NAMPT mAb was validated in a rat model of trauma (blast)/ventilator-induced lung injury (VILI). Sprague Dawley rats were challenged with trauma (blast)/VILI and intravenously (IV) injected with 100 μg an anti-NAMPT mAb (ALT-100) 30 minute following the blast. Rats, which were exposed to trauma (blast)/VILI and injected with vehicle, served as control. Lungs from the rats were then evaluated for injury after 4 hours of mechanical ventilation. Also, edema and inflammatory cell infiltration in lung tissue were assessed by hematoxylin and eosin (H&E) staining, as readout of lung injury. Results from this trauma (blast)/VILI lung injury model are provided in FIGS. 16A-C.

As shown in FIG. 16A, compared to non-challenged rats (FIG. 16A, inset in rightmost box), lung tissues from vehicle injected control trauma/VILI rats showed inflammatory cell infiltration and edema, which was indicative of trauma/VILI induced lung injury. In contrast, as shown in FIG. 16B, lung tissues from anti-NAMPT mAb treated trauma/VILI rats showed marked reduction in inflammatory cell infiltration and edema, thus indicating attenuation of trauma/VILI-induced lung injury by the anti-NAMPT mAb. The effect of the anti-NAMPT mAb on trauma/VILI-induced lung injury is summarized in FIG. 16C, which shows lung injury score of the rats, as assessed from the H&E staining indices. As shown in FIG. 16C, lung injury score was significantly reduced in rats that were treated with anti-NAMPT mAb compared to rats that were injected with vehicle control (p<0.05). Thus, the results outlined in FIGS. 16A-C show the efficacy of a NAMPT neutralizing mAb in attenuating trauma/VILI-induced lung injury.

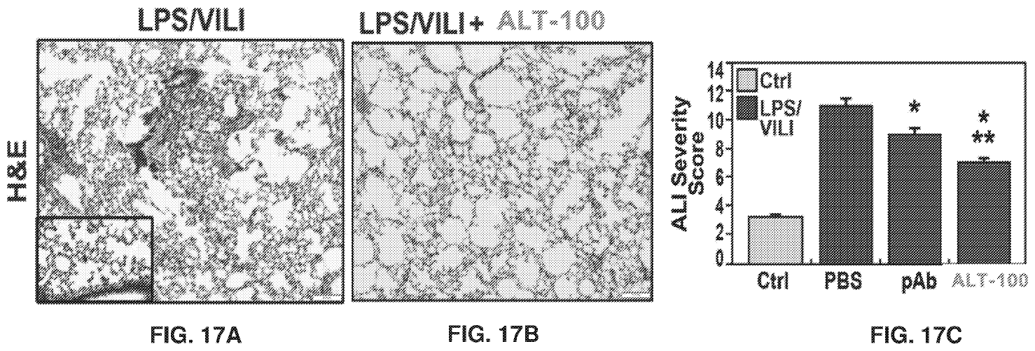

Next, the efficacy of the anti-NAMPT mAb was validated in a murine model of LPS/VILI. Mice were challenged with LPS for 18 hours followed by mechanical ventilation for 4 hours. Mice were injected with 10 μg (IV) of an anti-NAMPT mAb (ALT-100), an anti-NAMPT polyclonal antibody (pAb, IV), or vehicle control (PBS) 1 hour after LPS challenge. Mice, which were not exposed to LPS/VILI, served as control. Edema and inflammatory cell infiltration in lung tissue from the mice were then assessed by H&E staining, as readout of lung injury. Results from this LPS/VILI lung injury model are provided in FIGS. 17A-C.

As shown in FIG. 17A, compared to non-challenged mice (FIG. 17A, inset), lung tissues from vehicle injected control mice showed inflammatory cell infiltration and edema, which was indicative of LPS/VILI induced lung injury. In contrast, as shown in FIG. 17B, lung tissues from anti-NAMPT mAb treated mice showed marked reduction in inflammatory cell infiltration and edema, thus indicating attenuation of LPS/VILI-induced lung injury by the anti-NAMPT mAb. The effect of the anti-NAMPT mAb on trauma/VILI-induced lung injury is summarized in FIG. 17C, which shows acute lung injury (ALI) severity score of the mice, as assessed from the H&E staining indices. As shown in FIG. 17C, ALI was markedly reduced in mice treated with anti-NAMPT pAb or mAb compared to vehicle injected mice, with most robust reduction in ALI severity score observed in mice that were treated with the anti-NAMPT mAb (p<0.001). Thus, the results outlined in FIGS. 17A-C show the efficacy of a NAMPT neutralizing mAb in attenuating trauma/VILI-induced lung injury.

Accordingly, the results demonstrate the effectiveness of the anti-NAMPT mAb in reducing lung injury in pre-clinical in vivo lung injury models.

### Example 15

### Radiolabeled Anti-NAMPT Antibody Identifies Increased NAMPT Expression in Inflamed Lung Tissues

A humanized anti-NAMPT mAb was radiolabeled to develop an imaging probe that would be capable of non-invasively detecting NAMPT signaling pathway and NAMPT expression in different tissues in vivo. Considering the potentials of NAMPT as a diagnostic and/or prognostic biomarker in acute inflammatory conditions (e.g., COVID-19, ARDS and lung injury), the radiolabeled anti-NAMPT mAb could be used as a diagnostic tool in subjects who are at risk of developing such conditions, or for selecting subjects likely to respond to treatment of such inflammatory conditions with an anti-NAMPT mAb, including chronic conditions such as lung fibrosis, radiation injury, and cardiac fibrosis. The present example describes detection of NAMPT expression in inflamed tissues, such as LPS-challenged and ionizing radiation-exposed lungs, using the radiolabeled anti-NAMPT mAb.

First, to test the detection of NAMPT expression by the radiolabeled anti-NAMPT antibody, ^{99m}Tc-labeled anti-NAMPT mAb probe or radiolabeled IgG control Ab was injected into mice that were exposed to 20 Gy total lung irradiation (WTLI), and rapid autoradiograph imaging were performed.

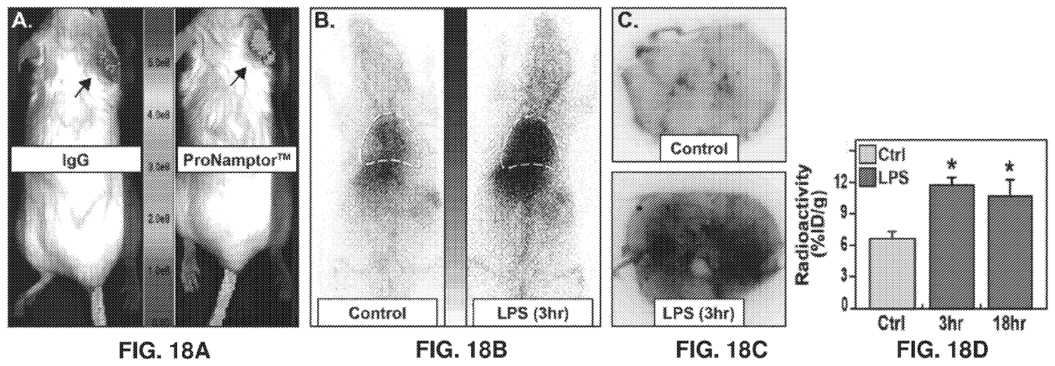

As shown in FIG. 18A, markedly higher radioactive uptake was observed in irradiated mice injected with radiolabeled anti-NAMPT mAb PRONAMPTOR (FIG. 18A, right panel) compared to irradiated mice injected with the radiolabeled IgG control (FIG. 18A, left panel). Thus, the results shown in FIG. 18A demonstrate the ability of the radiolabeled anti-NAMPT imaging probe in detecting radiation-induced NAMPT expression.

To further assess the detection of NAMPT expression by radiolabeled anti-NAMPT imaging probe, ^{99m}Tc-labeled anti-NAMPT mAb was injected into vehicle challenged control mice or LPS challenged mice 3 hours or 18 hours after LPS challenge, and rapid autoradiograph imaging was performed. Results from the analysis are shown in FIGS. 18B-D.

As shown in FIG. 18B, compared to control mice (FIG. 18B, left panel), LPS challenged mice showed markedly higher uptake of the radiolabeled anti-NAMPT imaging probe 3 hours after LPS challenge (FIG. 18B, right panel). Autoradiograph imaging of lungs from LPS challenged mice or control mice further confirmed this observation; compared to control mice (FIG. 18C, left panel), lungs of LPS challenged mice showed markedly higher uptake of the radiolabeled anti-NAMPT imaging probe 3 hour after LPS challenge (FIG. 18C, right panel). Moreover, as shown in FIG. 18D, compared to control mice, LPS challenged mice showed significantly higher radioactivity 3 hours and 18 hours after LPS challenge (p<0.05), indicating higher uptake of the radiolabeled anti-NAMPT imaging probe. Thus, the results described in FIGS. 18B-D demonstrate the ability of the radiolabeled anti-NAMPT imaging probe in detecting LPS-induced NAMPT expression.

Accordingly, the radiolabeled anti-NAMPT antibody was effective in detecting increased NAMPT expression in inflamed tissues. This underscores the potentials of utilizing the radiolabeled anti-NAMPT antibody as a tool for detection of NAMPT, which could be pivotal in using NAMPT as a diagnostic and/or prognostic biomarker in acute inflammatory conditions. Moreover, by virtue of detecting increased NAMPT expression in inflamed tissues, this radiolabeled anti-NAMPT imaging probe could be useful for selecting subjects who are likely to respond to treatment of acute inflammatory conditions with a neutralizing anti-NAMPT mAb.

### Example 16

### Expression of NAMPT in Human IPF

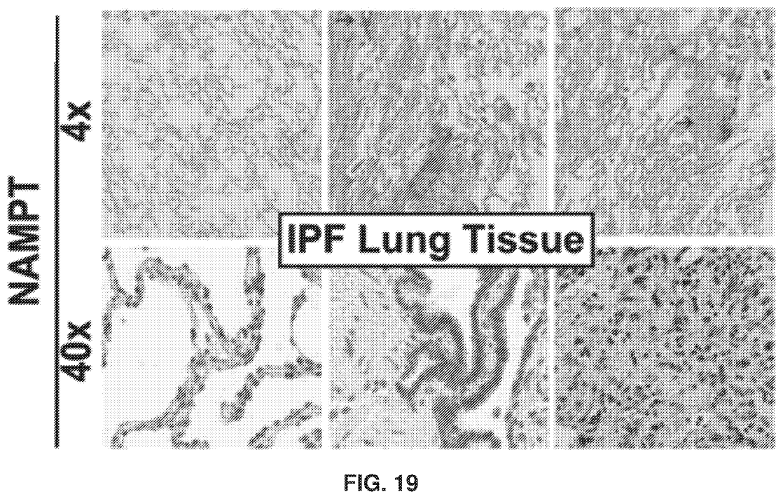

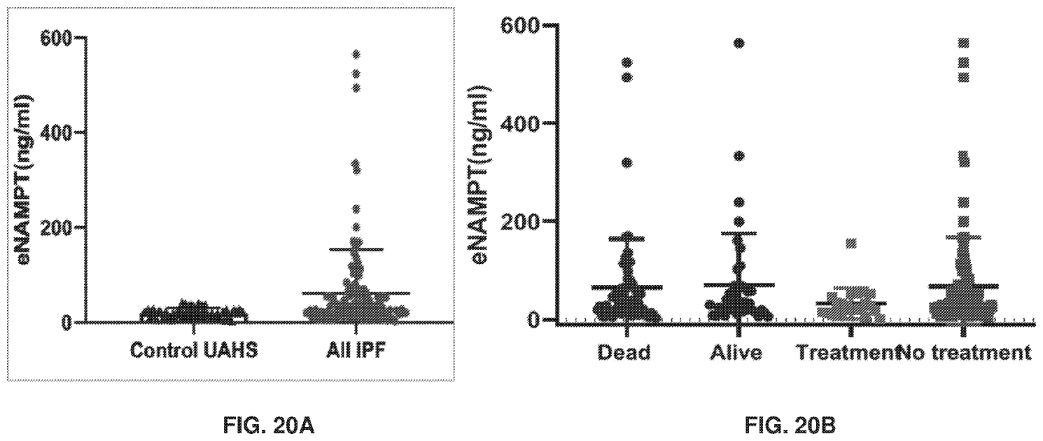

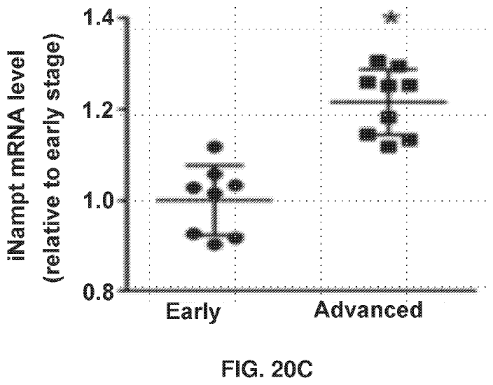

In order to assess the role of NAMPT in pulmonary fibrosis, expression of NAMPT was evaluated in lung tissues and plasma of idiopathic pulmonary fibrosis (IPF) patients. The results are shown in FIGS. 19 and 20A-C. To this end, lung tissues were isolated from patients with confirmed diagnosis of IPF and evaluated for NAMPT expression by immunohistochemical (IHC) staining. As shown in FIG. 19, NAMPT was found to be specifically expressed in fibroblasts within fibrotic regions of IPF lung tissue via IHC staining, thus indicating a role of NAMPT in pathophysiology of IPF. Next, plasma samples were obtained from IPF patients and healthy controls and expression of NAMPT was assessed by ELISA. As shown in FIG. 20A, plasma samples from IPF patients showed a marked increase in NAMPT level compared to that from healthy controls. To further assess NAMPT plasma levels in IPF, expression of NAMPT was evaluated in plasma samples from dead IPF patients, alive IPF patients, treated IPF patients and untreated IPF patients. As shown in FIG. 20B, no difference in NAMPT levels was evident between plasma samples from dead IPF patients, alive IPF patients, and untreated IPF patients. However, IPF patients who received treatment had markedly reduced NAMPT plasma levels, thus underscoring the role of NAMPT in IPF pathogenesis. The role of NAMPT in IPF pathogenesis and progression was further evaluated by assessing Nampt mRNA levels in fibroblasts isolated from advanced vs. early stage IPF patients. As shown in FIG. 20C, a significant increase in Nampt mRNA level was observed in fibroblasts from advanced IPF patients compared to those from early stage IPF patients (p<0.05), thus indicating increasing NAMPT expression with increasing IPF severity.

Hence, the results demonstrate a dysregulation of NAMPT expression and secretion in IPF, indicating a role for NAMPT in pathogenesis and progression of pulmonary fibrosis.

### Example 17

### Exploring the Role of NAMPT in IPF Using a Bleomycin-Induced Murine Lung Fibrosis Model

The role of NAMPT in IPF was further explored using a bleomycin-induced murine lung fibrosis model. To this end, NAMPT heterozygous (Nampt^{+/-}; “Nampt het”) mice or WT mice were challenged with bleomycin; WT and Nampt^{+/-} mice that were not challenged with bleomycin, served as controls. Lung fibrosis was assessed in the bleomycin-challenged groups and non-challenged control groups by evaluating soluble collagen in whole lungs.

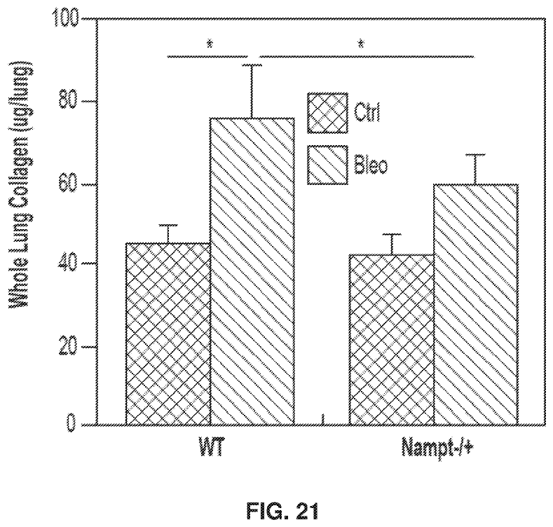

As shown in FIG. 21, bleomycin-challenged WT mice showed marked increase in lung fibrosis reflected by soluble collagen in whole lungs, compared to control WT mice (p<0.05). However, soluble collagen in whole lungs of bleomycin-challenged Nampt^{+/-} mice was significantly less than that from bleomycin-challenged WT mice (p<0.05), indicating that Nampt^{+/-} mice are protected from bleomycin-induced lung injury and lung fibrosis.

Thus, the results demonstrate proof-of-concept that in vivo targeting of Nampt leads to protection from lung fibrosis and underscore NAMPT as an effective therapeutic target in pulmonary fibrosis.

### Example 18

### Assessing NAMPT Expression and NAMPT SNPs in PAH Patients

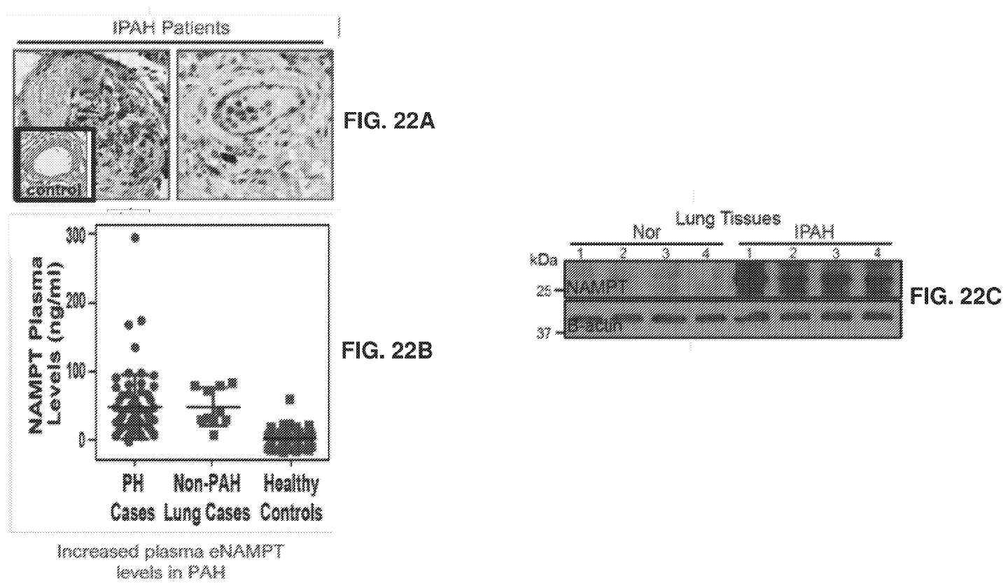

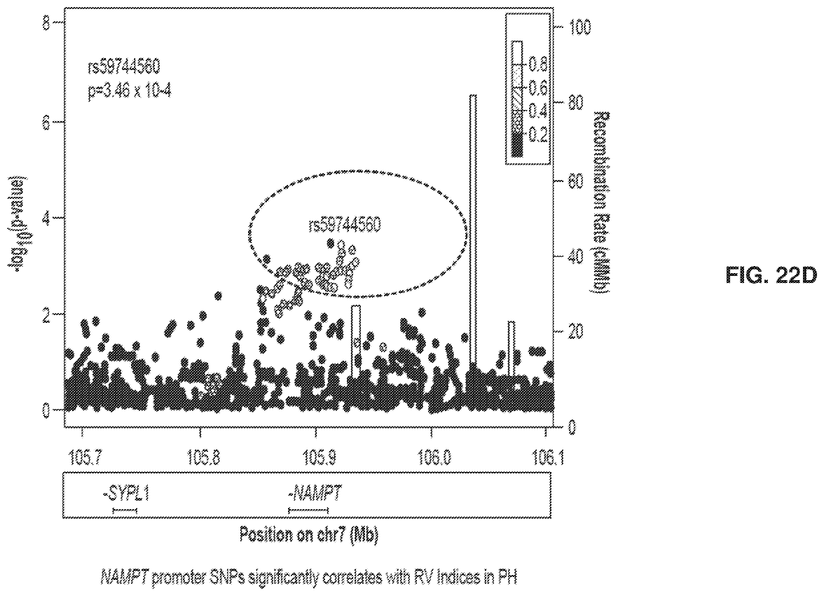

In order to assess the role of NAMPT in pulmonary arterial hypertension (PAH), expression of NAMPT was evaluated in lung tissues and plasma of patients with idiopathic pulmonary artery hypertension (IPAH). The results are shown in FIGS. 22A-C. As shown in FIG. 22A, lung tissue from IPAH patients showed marked increase in NAMPT expression compared to lung tissue from healthy control (FIG. 22A, inset). Next, plasma samples were obtained from patients with PAH, patients with non-PAH lung diseases, and healthy control subjects; NAMPT plasma levels were assessed by ELISA. The results are shown in FIG. 22B. While both PAH patients and non-PAH lung disease patients showed increased NAMPT plasma level compared to healthy controls, a marked increase in NAMPT plasma level was observed in patients with PAH. To further ascertain NAMPT expression in IPAH patients, lysates prepared from lung tissues of IPAH patients or normal healthy controls were subject to western blot analysis. As shown in FIG. 22C, a marked increase in NAMPT expression was observed in lung tissue from IPAH patients compared to lung tissues from healthy normal controls. Thus, the results demonstrate a dysregulation of NAMPT expression and secretion in PAH, indicating a role for NAMPT in PAH pathogenesis

Next, DNA from IPAH patients were analyzed to ascertain correlation between NAMPT promoter SNPs and right ventricular (RV) indices in a genome-wide association study (GWAS). As shown in FIG. 22D, the NAMPT promoter SNP rs59744560 is significantly correlated with RV indices, thus indicating the potential of using NAMPT promoter SNPs as genetic biomarkers of PAH, a predictor of PAH severity, and potentially a mechanism for identifying persistent PAH (pPAH) patients likely to responds to eNAMPT-neutralizing mAb therapy.

### Example 19

### Anti-NAMPT Antibody Reduces PAH Manifestation in a Rat Model

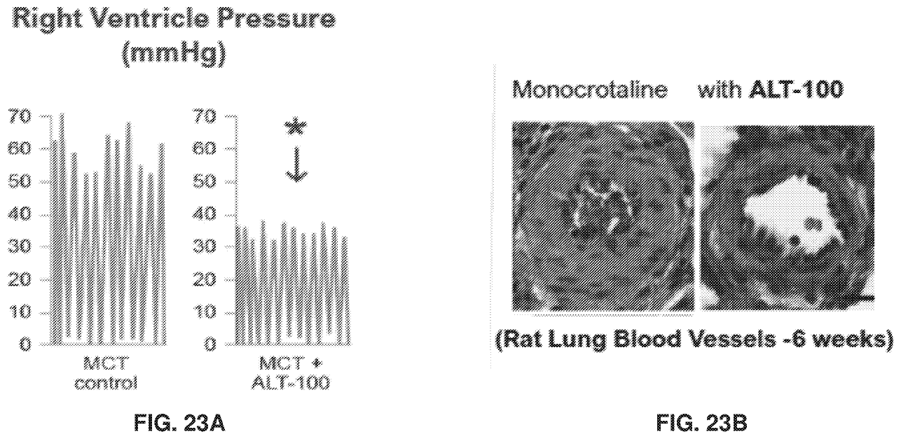

To explore the potentials of NAMPT as a therapeutic target in PAH, a rat monocrotaline (MCT) model of PAH was used. One dose of MCT (60 mg/kg body weight) was subcutaneously injected to Sprague-Dawley rats (190-200 g). The MCT-challenged rats were then injected with either an anti-NAMPT mAb (weekly, 100 μg/rat, intraperitoneal (i.p.)) or vehicle control (control MCT rats). The rats were then assessed for right ventricular systolic pressure (RVSP) and pulmonary artery remodeling. The results are shown in FIGS. 23A and 23B.

RVSP was determined in anti-NAMPT Ab treated MCT rats or control MCT rats by right heart catheterization using a Millar pressure transducer catheter. As shown in FIG. 23A, a significant decrease in RVSP was observed in MCT rats that were treated with anti-NAMPT mAb compared to control MCT rats (p<0.05).

Pulmonary artery remodeling was assessed using Aperio ImageScope software after lungs from anti-NAMPT mAb treated MCT rats or control MCT rats were stained with H&E. As shown in FIG. 23B, a marked decrease in pulmonary artery thickness was observed in anti-NAMPT mAb treated MCT rats compared to control MCT rats.

The results demonstrate that neutralization of NAMPT by anti-NAMPT mAb reverses vascular remodeling and RV dysfunction in a rat model of PAH, thus indicating the effectiveness of NAMPT as a therapeutic target in PAH.

What is claimed is:

1. A method of identifying a subject at risk of developing aggressive prostate cancer, comprising,
a) obtaining a sample from a subject having indolent prostate cancer; and
b) detecting the presence of at least one single nucleotide polymorphism (SNP) associated with human nicotinamide phosphoribosyl transferase (NAMPT) in the sample, wherein the SNP is selected from the group consisting of rs7789066, rs61330082, rs9770242, rs59744560, rs116647506, rs1319501, rs114382471, and rs190893183.

2. The method of claim 1, wherein the subject has indolent prostate cancer that is inherited.

3. The method of claim 1 or 2, wherein the subject has at least 2 SNPs, at least 3 SNPs, at least 4 SNPs, at least 5 SNPs, at least 6 SNPs, at least 7 SNPS, or at 8 SNPs selected from the group consisting of rs7789066, rs61330082, rs9770242, rs59744560, rs116647506, rs1319501, rs114382471, and rs190893183.

4. The method of claim 1 or 2, comprising detecting at least 2 SNPs selected from the group consisting of rs7789066, rs61330082, rs9770242, rs59744560, rs116647506, rs1319501, rs114382471, and rs190893183.

5. The method of any one of claims 1-4, comprising detecting at least one SNP selected from the group consisting of rs7789066, rs61330082, rs9770242 and rs59744560.

6. The method of any one of claims 1-5, comprising detecting at least one SNP selected from the group consisting of rs116647506, rs61330082, rs114382471, and rs190893183.

7. The method of any one of claims 1-6, wherein the subject is of African descent.

8. The method of any one of claims 1-7, wherein the detecting comprises using a polymerase chain reaction (PCR), a SNP microarray, SNP-restriction fragment length polymorphism (SNP-RFLP), dynamic allele-specific hybridization (DASH), primer extension (MALDI-TOF) mass spectrometry, single strand conformation polymorphism, and/or new generation sequencing (NGS).

9. The method of any one of claims 1-7, wherein the detecting comprises contacting the sample with an oligonucleotide probe that selectively hybridizes to a nucleotide sequence comprising the SNP, or a nucleotide sequence complementary thereto, and detecting selective hybridization of the oligonucleotide probe.

10. The method of claim 9, wherein the oligonucleotide probe comprises a detectable label, and wherein detecting selective hybridization of the probe comprises detecting the detectable label.

11. The method of claim 10, wherein the detectable label comprises a fluorescent label, a luminescent label, a radionuclide, or a chemiluminescent label.

12. The method of claim 9, wherein the oligonucleotide probe comprises a bilabeled oligonucleotide probe, comprising a fluorescent moiety and a fluorescent quencher.

13. The method of any one of claims 1-7, further comprising detecting one or more additional SNPs associated with a NAMPT promoter activity level that is higher than a baseline NAMPT promoter activity level.

14. The method of any one of claims 1-13, wherein the sample is a plasma sample.

15. A method of treating a subject having indolent prostate cancer, said method comprising:
a) obtaining a sample from a subject having indolent prostate cancer;
b) detecting the presence or absence of at least one SNP in the sample, wherein the SNP is selected from the group consisting of rs7789066, rs61330082, rs9770242, rs59744560, rs116647506, rs1319501, rs114382471, and rs190893183, and wherein the presence of the at least one SNP indicates that the subject is at risk for developing aggressive prostate cancer; and
c) administering to the subject at risk for developing aggressive prostate cancer (i) an effective amount of an eNAMPT inhibitor and/or (ii) one or more of radiation therapy (e.g., external beam radiation; and/or brachytherapy); hormone therapy such as luteinizing hormone-releasing hormone (LH-RH) agonists (e.g., leuprolide; goserelin; triptorelin; and/or histrelin) or other medications to stop the body from producing testosterone (e.g., ketoconazole; and/or abiraterone); anti-androgens (e.g., bicalutamide; nilutamide; flutamide; and/or enzalutamide); chemotherapy; and biological therapy (e.g., sipuleucel-T),
such that the subject having indolent prostate cancer is treated.

16. The method of claim 15, wherein the sample is a plasma sample.

17. The method of claim 15 or 16, comprising detecting at least 2 SNPs, at least 3 SNPs, at least 4 SNPs, at least 5 SNPs, at least 6 SNPs, at least 7 SNPS, or 8 SNPs selected from the group consisting of rs7789066, rs61330082, rs9770242, rs59744560, rs116647506, rs1319501, rs114382471, and rs190893183.

18. The method of any one of claims 15-17, wherein the SNP is selected from the group consisting of rs7789066, rs61330082, rs9770242 and rs59744560.

19. The method of any one of claims 15-18, wherein the SNP is selected from the group consisting of rs116647506, rs61330082, rs114382471, and rs190893183.

20. The method of any one of claims 15-19, wherein the subject is of African descent.

21. The method of any one of claims 15-20, wherein the detecting comprises using a polymerase chain reaction (PCR), a SNP microarray, SNP-restriction fragment length polymorphism (SNP-RFLP), dynamic allele-specific hybridization (DASH), primer extension (MALDI-TOF) mass spectrometry, single strand conformation polymorphism, and/or new generation sequencing (NGS).

22. The method of any one of claims 15-21, wherein the presence of the SNP is determined by contacting the sample with an oligonucleotide probe that selectively hybridizes to a nucleotide sequence comprising the SNP, or a nucleotide sequence complementary thereto, and detecting selective hybridization of the oligonucleotide probe.

23. The method of claim 22, wherein the oligonucleotide probe comprises a detectable label, and wherein detecting selective hybridization of the probe comprises detecting the detectable label.

24. The method of claim 23, wherein the detectable label comprises a fluorescent label, a luminescent label, a radionuclide, or a chemiluminescent label.

25. The method of claim 22, wherein the oligonucleotide probe comprises a bilabeled oligonucleotide probe, comprising a fluorescent moiety and a fluorescent quencher.

26. The method of any one of claims 15-25, further comprising detecting one or more additional SNPs associated with a NAMPT promoter activity level that is higher than a baseline NAMPT promoter activity level.

27. The method of claim 13 or 26, wherein the baseline NAMPT promoter activity level is a level associated with indolent prostate cancer.

28. The method of any one of claims 15-27, comprising administering the eNAMPT inhibitor, wherein the eNAMPT inhibitor is an anti-eNAMPT antibody.

29. The method of claim 28, wherein the anti-eNAMPT antibody comprises a heavy chain comprising a variable region comprising CDR1, CDR2, and a CDR3 domains as set forth in amino acid sequences of SEQ ID Nos: 4, 5, and 6, respectively; and a light chain comprising a variable region comprising CDR1, CDR2, and a CDR3 domains as set forth in amino acid sequences of SEQ ID Nos: 7, 8, and 9, respectively.

30. The method of claim 29, wherein the heavy chain variable region comprises the amino acid sequence set forth in SEQ ID NO: 2, and the light chain variable region comprises the amino acid sequence set forth in SEQ ID NO: 3.

31. The method of claim 28, wherein the anti-eNAMPT antibody comprises a heavy chain comprising a variable region comprising CDR1, CDR2, and a CDR3 domains as set forth in amino acid sequences of SEQ ID Nos: 12, 13, and 14, respectively; and a light chain comprising a variable region comprising CDR1, CDR2, and a CDR3 domains as set forth in amino acid sequences of SEQ ID Nos: 15, 16, and 17, respectively.

32. The method of claim 31, wherein the heavy chain variable region comprises the amino acid sequence set forth in SEQ ID NO: 10, and the light chain variable region comprises the amino acid sequence set forth in SEQ ID NO: 11.
